(19) United States
(12) Patent Application Publication
(10) Pub. No.: US 2002/0006915 A1
(43) Pub. Date: Jan. 17, 2002

(54) Use of COX-2 inhibitors to treat sepsis, complications thereof, and EP receptor modulation

(76) Inventors: Vivian E. Mack Strong, New York, NY (US); Philip P. Stapleton, New York, NY (US); John M. Daly, Pelham Manor, NY (US)

Correspondence Address: Michael L. Goldman, Esq., NIXON PEABODY LLP, Clinton Square, P.O. Box 31051, Rochester, NY, 14603, US

(21) Appl. No.: 09/782,936

(22) Filed: Feb. 14, 2001

Related U.S. Application Data
(60) Provisional application No. 60/182,524, filed on Feb. 15, 2000.

(51) Int. Cl. A61K 48/00; A61K 38/00; A61K 31/415

(52) U.S. Cl. 514/44; 514/2; 514/406

(57) ABSTRACT

The present invention is directed to methods of preventing, inhibiting, reversing and/or ameliorating complications in those having or at risk for systemic inflammatory response syndrome, e.g., sepsis, including multiple organ dysfunction syndrome, pancreatitis, burns, trauma, and complications of sepsis such as bacteremia, pneumonia, urinary tract infections, wound infections, and drug reactions. The methods comprise administration of an effective amount of at least one of a selective inhibitor of cyclooxygenase-2, a drug which stimulates one or more PGE₂ receptors or a drug which interferes with binding of PGE₂ to one of more PGE₂ receptors.

This application was originally filed as provisional application Ser. No. 60/182,524 on Feb. 15, 2000.

The invention described herein was made in the course of and under National Institutes of Health Grant RO1DK5O201 and is therefore subject to the rights of the U.S. government.

### FIELD OF THE INVENTION

The present invention is directed to the prevention and treatment of patients at risk for, or having, systemic inflammatory response syndrome and septic complications. The present invention therefore pertains to treatment of patients in the clinical settings of shock, sepsis, trauma, major elective surgery and critical care.

### BACKGROUND OF THE INVENTION

Trauma leads to impairments in immunity by initiating an inflammatory response that predisposes the host to infectious complications. [1] For injured patients who develop infectious sequelae, the consequences are often fatal. In the United States, more than 500,000 patients develop sepsis per year and only 55 to 65% survive. [2, 3]

It is well documented that even after stabilizing patients after serious injury, such as those incurred by motor vehicle accidents, burns, or life threatening blood loss from penetrating injury, those who survive for greater than 24 hours are most likely to die from infections that occur late following injury. In fact, of patients who survive the first 24 hours after serious injury, greater than 75% die as a result of late infectious complications such as pneumonia, generalized sepsis or multiple organ dysfunction; all processes related to a compromised immune system. [4]

Although numerous studies have demonstrated that injury induces a state of immunosuppression, the precise mechanisms are still under investigation prostaglandin receptors EP2 and EP4 are down regulated and that reversing this down regulation of receptor subtypes markedly improves survival.

The present invention provides methods of treating patients who have undergone shock, trauma, or other injury (including surgical insult) and who are at risk for developing infectious complications such as systemic inflammatory response syndrome. In one embodiment, the method comprises administering to a patient an effective amount of a selective inhibitor of cyclooxygenase-2. In another embodiment, the method comprises administering an effective amount of a drug which either stimulates one or more PGE₂ receptors (e.g., EP1, EP2, EP3, or EP4 receptor subtypes) or interferes with binding of PGE₂ to one or more of the PGE₂ receptor subtypes. The present invention therefore provides therapeutic interventions which selectively modulate the immune response after trauma, reduce the incidence of infectious complications and improve survival after traumatic injury.

### SUMMARY OF THE INVENTION

The present invention provides methods of prophylaxis of a patient at risk for systemic inflammatory response syndrome and complications thereof or of treating a patient having systemic inflammatory response syndrome or complications thereof. Systemic inflammatory response syndrome may include at least one of sepsis, pancreatitis, burns, or trauma. Complications of systemic inflammatory response syndrome may include at least one of septic shock, infections such as pneumonia, septicemia, bacteremia, urinary tract infections, wound infections or drug reactions.

Patients at risk for systemic inflammatory response syndrome and complications thereof include, e.g., patients who have sustained at least one of trauma, burn injury, life threatening blood loss from penetrating injury, or patients who have undergone surgery.

In one embodiment of the invention, the method comprises administering to a patient a therapeutically effective amount of an inhibitor of cyclooxygenase-2. Preferably, a specific inhibitor of cyclooxygenase-2 is administered. Examples of cyclooxygenase-2 inhibitors useful in the methods of the present invention include but are not limited to NS-398, celicoxib, MK-0966, and paracoxib.

In another embodiment of the invention, the method comprises administering to a patient a therapeutically effective amount of a drug which interferes with binding of PGE₂ to one or more PGE₂ receptors i.e., at least one of EP1, EP2, EP3, and EP4.

In yet another embodiment of the invention, the method comprises administering to a patient a therapeutically effective amount of a drug which stimulates one or more PGE₂ receptors.

Examples of compositions which function to interfere with binding of PGE₂ to one or more PGE₂ receptors or which function to stimulate one or more PGE₂ receptors include small molecules, peptides, peptide mimetics, and RNA-DNA-based structures.

The present invention further provides a method of beneficial immune modulation which comprises administering to a patient in need of such modulation a therapeutically effective amount of a drug which either stimulates one or more PGE₂ receptors or which interferes with binding of PGE₂ to one or more PGE₂ receptors. Such drugs may include for example, small molecules, peptides, peptide mimetics, and RNA-DNA-based structures.

Examples of drugs which may be used to interfere with binding of PGE₂ to one or more PGE₂ receptors include AH-6809, ONO-8711, ONO-8713, and AH23848. Examples of drugs which may be used to stimulate one or more PGE₂ receptors include sulprostone, 11-deoxy-PGE₁, or ONO-AP-324.

### BRIEF DESCRIPTION OF THE DRAWINGS

FIG. 1 graphically depicts the effects of 3 doses in vivo of NS-398 injections given within 24 hours of injury on PGE₂ production from LPS stimulated splenic macrophages shown at 7 days following injury. Groups include: sham+vehicle (control); sham+NS-398; trauma+vehicle; and trauma+NS-398. Each value represents the average of n=8 mice per group±SEM. There is a significant difference in control versus trauma values of *p<0.04 and a significant difference in PGE₂ production of T versus TN values of **p<0.03. Experiment shows representative data of one experiment (repeated three times).

FIG. 2 graphically depicts the effects of 3 doses in vivo of NS-398 injections given within 24 hours of injury on IL-6 production from LPS stimulated splenic macrophages at 7 days following injury. Groups are as described for FIG. 1. Each value represents the average of n=8 mice per group±SEM. There is a significant difference in control versus trauma values of *p<0.02 and a significant difference in cytokine production of T versus TN values of **p<0.02. Experiment shows representative data of one experiment (repeated three times).

FIG. 3 graphically depicts the effects of 3 doses in vivo of NS-398 injections given within 24 hours of injury on TNF-α production from LPS stimulated splenic macrophages at 7 days following injury. Groups are as described for FIG. 1. Each value represents the average of n=8 mice per group±SEM. There is a significant difference in control versus trauma values of *p<0.001 and a significant difference in cytokine production of T versus TN **p<0.05. Experiment shows representative data of one experiment (repeated three times).

FIG. 4 graphically depicts the effects of 3 doses in vivo of NS-398 injections given within 24 hours of injury on NO production from LPS+IFN-γ stimulated splenic macrophages at 7 days following injury. Groups are as described for FIG. 1. Each value represents the average of n=8 mice per group±SEM. There is a significant difference in control versus trauma values of *p<0.001 and a significant difference in NO production of T versus TN **p<0.05. Experiment shows representative data of one experiment (repeated three times).

FIG. 5 is a photographic representation of a gel showing the relative levels of COX-2 mRNA expression in mice at 7 days after 3 doses in vivo of NS-398 injections given within 24 hours of injury compared to the constituitively expressed levels of GAPDH, a housekeeping gene. The four groups tested were: sham+placebo (control, C); sham+NS-398 (control+NS-398, CN); trauma+placebo (trauma); and trauma+NS-398 (TN).

FIG. 6 is a Kaplan-Meier survival plot showing the effects of 3 doses in vivo of NS-398 injections given during the first 24 hours following injury on subsequent survival after giving a septic challenge. Groups included; sham+vehicle (control); sham+NS-398 (control+398); trauma+vehicle (trauma); and trauma+NS-398. Each value represents the average of n=10-11 mice per group.

FIG. 7 graphically depicts the effect of twice a day in vivo dosing of NS-398 over 7 days on PGE₂ production, determined from LPS stimulated splenic macrophages at 7 days after trauma. Data represents average of n=8 mice per group±SEM, repeated three times. *P<0.05 for control versus trauma. **P<0.05 for trauma versus trauma+NS-398. Groups are as described for FIG. 6.

FIG. 8 graphically depicts the effect of twice a day in vivo dosing of NS-398 over 7 days on the lipooxygenase pathways via production of LTB₄, determined from LPS stimulated splenic macrophages at 7 days after trauma. Groups, sample size, and P values are as for FIG. 7.

FIG. 9 graphically depicts the effect of twice a day in vivo dosing of NS-398 over 7 days on the pro-inflammatory mediator TNF-α, determined from LPS stimulated splenic macrophages at 7 days after trauma. Data represents average of n=8 mice per group±SEM, repeated three times. *P<0.05 for control versus trauma. **P<0.05 for trauma versus trauma+NS-398. Groups are as described for FIG. 6.

FIG. 10 graphically depicts the effect of twice a day in vivo dosing of NS-398 over 7 days on the pro-inflammatory mediator NO, determined from LPS stimulated splenic macrophages at 7 days after trauma. Groups are as described for FIG. 6. Sample size and P values are as for FIG. 9.

FIG. 11 graphically depicts the effect of twice a day in vivo dosing of NS-398 over 7 days on the pro-inflammatory mediator IL-6, determined from LPS stimulated splenic macrophages at 7 days after trauma. Sample size and P values are as for FIG. 9.

FIG. 12 is photographic reproduction of an mRNA gel blot showing the effect of NS-398 on COX-2 mRNA levels in splenic macrophages compared to constitutively expressed levels of GAPDH, a housekeeping gene. Data is from macrophages pooled from 3 mice per group and is representative of data repeated 3 times. C: sham+placebo, CN: sham+NS-398, T: trauma+placebo, TN: trauma+NS-398.

FIG. 13 is a photographic representation of a Western blot showing the effect of twice a day in vivo dosing of NS-398 over 7 days on cystolic IκB-α levels and activation of nuclear NF-κB. Results are from pooled macrophages of 3 mice per group and represent typical gel and Western blot repeated three times. C: sham+placebo, CN: sham+NS-398, T: trauma+placebo, TN: trauma+NS-398.

FIG. 14 graphically depicts the effects of twice a day in vivo dosing of NS-398 over 7 days on survival of mice following septic challenge 7 days after trauma. Survival plots represent n=10-12 mice per group and all animals were followed over 10 days before being euthanized. *P<0.001 for trauma versus trauma+NS-398 group.

FIG. 15 graphically depicts PGE₂ production in pg/mL from LPS (10 ng/mL) stimulated PBMC's±co-incubation with NS-398, represented as the mean±SEM from 9-10 subjects per group. Groups include C=control; CN=control+10 μM NS-398; T=trauma; TN=trauma+10 μM NS-398.

FIG. 16 is a photographic representation of an RT-PCR gel demonstrating decreased mRNA expression levels of EP2 and EP4 in injured patients which is restored after ex vivo treatment with NS-398. Decreased expression of EP2 and EP4 is also shown in PBMCs co-incubated with 100 μM PGE₂. Control PBMC's, PBMC's of injured patients, C+NS: control+NS-398, I+NS: Injury+NS-398, C+PGE₂: Control+1 μM PGE₂: C+PGE₂: control+100 μM PGE₂.

FIG. 17 graphically depicts LTB₄ production in pg/mL from LPS (10 ng/mL) stimulated PBMC's±co-incubation with NS-398, represented as the mean±SEM from 9-10 subjects per group. Group abbreviations are as in FIG. 16.

FIG. 18 graphically depicts TNF-α production in pg/mL from LPS (10 ng/mL) stimulated PBMC's±co-incubation with NS-398, represented as the mean±SEM from 9-10 subjects per group. Group abbreviations are as in FIG. 15.

FIG. 19 is a photographic representation of a gel photograph showing verification of EP1 and EP3 primer integrity via RT-PCR. Lanes are as follows; EP1−=EP1 probed unstimulated sample, EP1+=EP1 probed LPS (10 ng/mL) stimulated sample, EP3−=EP3 probed unstimulated sample, EP3+=EP3 probed LPS stimulated sample. EP3 band was confirmed as present in a renal cell carcinoma cell line.

FIG. 20 is a photographic representation of a gel run with a representative sample of data from 10 separate patients, with results confirmed in 9/10 patients. Bands correlate with expression of EP2 receptors within 12 hours after injury±NS-398 co-culture from LPS stimulated human PBMC's via RT-PCR. Lanes are as follows: C=control; CN=control+NS-398 co-culture; T=trauma; TN=trauma+NS-398 co-culture.

FIG. 21 is a photographic representation of a gel run with a representative sample of data run from 10 separate patients, with results confirmed in 9/10 patients. Bands correlate with expression of EP4 receptors within 12 hours after injury from LPS stimulated human PBMC's via RT-PCR. Lanes are as follows: C=control; CN=control+NS-398 co-culture; T=trauma; TN=trauma+NS-398 co-culture.

FIG. 22A is a photographic representation of a gel run with LPS stimulated human PBMC's coincubated with PGE₂. Shown is the effect of increasing doses of PGE₂ on the expression of EP2 receptors. Lanes are as follows; C−=unstimulated control; C+=LPS stimulated control; 10⁻⁴=stimulated control co-incubated with 10⁻⁴ μM PGE₂; 10⁻²=stimulated control co-incubated with 10⁻² μM PGE₂; 10=stimulated control co-incubated with 10 μM PGE₂; 100=stimulated control co-incubated with 100 μM PGE₂.

FIG. 22B is a photographic representation of a gel run with LPS stimulated human PBMC's coincubated with PGE₂. Shown is the effect of increasing doses of PGE₂ on the expression of EP4 receptors. Lanes are as described in FIG. 22A.

FIG. 23 is a photographic representation of a gel showing expression of CD14 receptors via RT-PCR from stimulated human PBMC's and effect of co-culture with 100 μM PGE₂. The gel is a representative sample of data run from 10 separate patients. Lanes are as follows; C+=stimulated control; C+PGE₂=stimulated control co-cultured with PGE2; T+=stimulated trauma; T+PGE2=stimulated trauma co-cultured with 100 μM PGE₂.

FIG. 24 is photographic representation of a gel showing expression of COX-2 versus GAPDH via RT-PCR from LPS stimulated human PBMC's from control and injured subjects co-cultured+/−NS-398. The gel is a representative sample of data run from 10 separate patients, with results confirmed in 9/10 patients. Lanes are as follows; C=stimulated control; CN=stimulated control co-cultured with NS-398; T=stimulated trauma; TN=stimulated trauma co-cultured with NS-398.

### DETAILED DESCRIPTION OF THE INVENTION

The present invention is based on the discovery that the increase in circulating prostaglandins which occurs early after trauma or injury, may be blocked by administering a COX-2 inhibitor during the first 24 hours post-injury and results in markedly improved survival.

In connection with the present invention, it has also been discovered that after serious injury, the prostaglandin receptors EP2 and EP4 are down regulated. The present invention therefore also contemplates administering a drug which reverses down regulation of one or more PGE₂ receptors (EP1, EP2, EP3 and EP4), in order to increase survival.

Thus, in accordance with the methods of the present invention, the increase in circulating prostaglandins and/or the down regulation of receptor subtypes in a patient suffering trauma or other injury, is prevented, inhibited, reversed and/or ameliorated.

In one aspect of the invention, there is provided a method of prophylaxis of a patient at risk for systemic inflammatory response syndrome and complications thereof or of treating a patient having systemic inflammatory response syndrome or complications thereof. The method comprises administering to the patient a therapeutically effective amount of an inhibitor of cyclooxygenase-2. Preferably, a selective inhibitor or cyclooxygenase-2 is used.

In another aspect of the invention, there is provided a method of prophylaxis of a patient at risk for systemic inflammatory response syndrome and complications thereof or of treating a patient having systemic inflammatory response syndrome or complications thereof. The method comprises administering to the patient a therapeutically effective amount of a drug which either stimulates one or more PGE₂ receptors (i.e., subtypes EP1, EP2, EP3, or EP4) or which interferes with binding of PGE₂ to one or more of the PGE₂ receptors.

In still another aspect of the invention, there is provided a method of beneficial immune modulation which comprises administering to a patient in need of such modulation a therapeutically effective amount of a drug which stimulates one or more PGE₂ receptors or which interferes with binding of PGE₂ to one or more PGE₂ receptors.

The term “systemic inflammatory response syndrome” is used herein to mean a condition that can trigger an acute inflammatory reaction, the systemic manifestations of which are associated with release into the bloodstream of a large number of endogenous mediators of inflammation and includes, for example, conditions such as sepsis, pancreatitis, burns, and trauma. The term “sepsis” is used to mean a serious infection, localized or bacteremic, that is accompanied by systemic manifestation of inflammation and includes multiple organ dysfunction syndrome (MODS). The complications referred to include, for example, shock including septic shock which is sepsis with hypoperfusion and hypotension refractory to fluid therapy, infections such as pneumonia, septicemia, bacteremia, urinary tract infections, wound infections, and drug reactions.

The term “cyclooxygenase-2 inhibitor” or “COX-2 inhibitor” is used herein to mean any compound that binds to cyclooxygenase-2 enzyme and stops it from functioning.

The term “selective inhibitor of cyclooxygenase-2” or “selective COX-2 inhibitor” is used herein to mean a compound which selectively inhibits cyclooxygenase-2 in preference to cyclooxygenase-1 and particularly a compound for which the ratio of the IC₅₀ concentration (concentration inhibiting 50% of activity) for cyclooxygenase-1 to the IC₅₀ concentration for cyclooxygenase-2 is greater than 1. Such ratio is readily determined by assaying for cyclooxygenase-2 activity and assaying for cyclooxygenase-1 activity by the method set forth at column 39, line 55 through column 40, line 36 of Talley et al., U.S. Pat. No. 5,633,272, which is incorporated herein by reference, and from the resulting data obtaining a ratio of IC₅₀.

There are many COX-2 inhibitors and many selective COX-2 inhibitors. Any COX-2 inhibitor or selective COX-2 inhibitor may be used in the methods of the present invention.

Those referred to as being “at risk” include, for example, patients with a major injury or trauma leading to impaired immune response and increased susceptibility to infectious complications. Examples include patients who have been in motor vehicle accidents, patients who have sustained burns, or patients who have sustained life threatening blood loss from penetrating injury, and surgical patients.

The selective inhibitors of cyclooxygenase-2 useful for practicing the present invention are preferably those where the ratio of the IC₅₀ concentration for cyclooxygenase-1 to the IC₅₀ concentration for cyclooxygenase-2 is 5 or more. Even more preferred are selective inhibitors of cyclooxygenase where the ratio of the IC₅₀ concentration for cyclooxygenase-1 to the IC₅₀ concentration for cyclooxygenase-2 is 100 or more.

Selective inhibitors of cyclooxygenase-2 and methods of preparation thereof are set forth, for example, in WO99/30721, the whole of which is incorporated herein by reference.

Another selective inhibitor of cyclooxygenase-2 is NS 398 which is N-(2-cyclohexyloxy-4-nitrophenyl)methanesulfonamide, commercially available from Cayman Chemical of Ann Arbor, Mich.

Preferred selective inhibitors of cyclooxygenase-2 for use herein include 4-[5-(4-methylphenyl)-3-(triluoromethyl)-1H-pyrazol-1-yl]benzenesulfonamide which is compound (4) of WO99/30721 and is denoted celicoxib (trade name Celebrex®) and 3-(phenyl)-4-(4-(methylsulfonyl)phenyl)-2-(5H)-furanone which is compound (63) of WO99/30721 and is denoted MK-0966 and Vioxx®.

The dosage of selective inhibitor of cyclooxygenase-2 for the method herein is a cyclooxygenase-2 inhibiting amount which is a therapeutically effective amount. The precise therapeutically effective amount of a cyclooxygenase-2 inhibitor to be used in the methods of the present invention can be determined by the ordinarily skilled artisan with consideration of individual differences in age, weight, and condition of the patient.

In general, the dosage for any particular agent will vary within the range. For compound (4) referred to above, the dosage preferably ranges from about 3 to about 15 mg/kg, e.g., and is preferably about 12 mg/kg. For compound (63) referred to above, the dosage normally ranges from about 12.5 to about 50 mg daily.

Paracoxib is another cyclooxygenase-2 inhibitor which may be administered intravenously to a patient at a dosage levels in the range recommended by the manufacturer.

A drug which interferes with binding of PGE₂ to one or more PGE₂ receptor subtypes i.e., an antagonist, may include for example, a ligand such as a small molecule, peptide, peptide mimetic, or RNA-DNA-based structure. Examples of drugs which interfere with binding of PGE₂ to the EP1 receptor include but are not limited to: AH-6809 (available from Biomol, Plymouth Meeting, Pa.), SC-19220, SC-53122 (Biomol), ONO-8711 (available from ONO Pharmaceuticals, Osaka, Japan), and ONO-8713 (ONO Pharmaceuticals). An example of a drug which interferes with binding of PGE₂ to the EP4 receptor includes but is not limited to AH23848 (available from Glaxo Wellcome, UK).

A drug which stimulates one or more of the different EP receptor subtypes, i.e., an agonist, may include for example, a ligand such as a small molecule, peptide, peptide mimetic, or RNA-DNA-based structure. An example of a drug which stimulates the EP1 receptor includes, but is not limited to sulprostone (available from Cayman Chemical Co., Ann Arbor, Mich.). An example of a drug which stimulates the EP2 receptor includes, but is not limited to 11-deoxy-PGE₁ (Cayman Chemical Co.) An example of a drug which stimulates the EP3 receptor includes, but is not limited to ONO-AP-324 (ONO Pharmaceuticals). An example of a drug which stimulates the EP4 receptor includes, but is not limited to 11-deoxy-PGE₁ (Cayman Chemical Co.).

The dosage of drug which interferes with binding of PGE₂ to one or more receptor subtypes is an amount which will interfere with binding which is a therapeutically effective amount. The dosage of drug which stimulates one or more receptor subtypes is an amount which stimulates one or more receptors, which is a therapeutically effective amount. The precise therapeutically effective amount of antagonist or agonist to be used in the methods of the present invention can be determined by the ordinarily skilled artisan with consideration of individual differences in age, weight, condition of the patient, and type and concentration of antagonist or agonist.

A cyclooxygenase-2 inhibitor, PGE₂ receptor antagonist, or PGE₂ receptor agonist, may be administered in any way which is medically acceptable which may depend on the type of injury or condition being treated. Possible administration routes such as intravascular, intravenous, intraaterial, subcutaneous, intramuscular, intraperitoneal, intraventricular, intraepidural or others as well as oral, nasal, ophthalmic, rectal, topical or inhalation. A cyclooxygenase-2 inhibitor, PGE₂ receptor antagonist, or PGE₂ receptor agonist, may also be applied to tissue surfaces during surgery.

The route of administration is preferably systemic, e.g., oral or parenteral, e.g., intravenous.

Administration is preferably started within 24 hours, very preferably within 12 hours, after injury or burn or trauma or infection detection or admittance into an intensive care unit as a result thereof. Thus for example, after a traumatic injury, a patient may be administered a COX-2 inhibitor, PGE₂ receptor antagonist, or PGE₂ receptor agonist, as soon as possible within the aforementioned time frame and after the patient is stabilized.

The invention is further illustrated by the following examples which are not intended in any way to limit the scope of the invention.

### EXAMPLE I

### Blocking Prostaglandin E₂ after Trauma Attenuates Pro-inflammatory Cytokines and Improves Survival

### Materials and Methods

Animals

Inbred, 6-8 week old female BALB/c mice (weight 19-22g) were purchased from Charles River Laboratories, Wilmington, Mass. On arrival at the housing facility, animals were acclimatized for 2-4 days prior to experiments and allowed food and water ad libitum. This facility has been approved by the American Association for Accreditation of Laboratory Animal Care, and the Institutional Animal Care and Use Committee of the Weill Medical College of Cornell University.

Experimental Protocol

Mice were randomly assigned to receive either anesthesia alone (control) or anesthesia and a femur fracture plus 40% hemorrhage (trauma). Animals were anesthetized with inhaled methoxyflurane (Pitman-Moore Inc, Mundelein, Ill.). The right femur of each mouse in the trauma group was fractured by making a 0.5 cm incision of the ventral surface of the mouse in the inguinal region. The intermuscular plane was dissected to expose the proximal one-third of the femur, which was then fractured with a sterile scissors. The wound was closed in a single layer with 3.0 polyglycolic acid sutures after hemostasis was ensured. The 40% hemorrhage was via a standard retro-orbital approach with a heparinized microcapillary tube. An approximate 10% mortality of traumatized mice was observed within 30 minutes of the procedure. Immediately following injury, animals were injected with NS-398 or 4% DMSO vehicle, and injected again at 12 and 24 hours after injury. All of these control and traumatized animals survived until they were euthanized via CO₂ asphyxiation. Four groups, sham+placebo (C), sham+NS-398 (CN), trauma+placebo (T), or trauma+NS-398 (TN) were studied. In a separate experiment, mice were randomized as above into the four groups (n=10-11/group), and on day 7 after injury, given a septic challenge by cecal ligation and puncture (CLP). This procedure was performed via a sterile 0.5 cm midline abdominal incision, after which the bowel was gently mobilized to expose the cecum and a 4.0 silk ligature was placed around a portion of the ileocecal region. Following this, a 26 gauge needle was used to puncture the portion distal to the ligature and a small amount of expressed stool was visualized. The midline incision was then reapproximated using 4-0 polyglycolic acid sutures and animals followed for survival over 24 days before sacrifice.

NS-398 Preparation

NS-398 (N-[2-(cyclohexyloxy)-4-nitrophenyl]-methanesulfonamide) was obtained from Cayman Chemical, Ann Arbor, Mich., and was prepared in a 4% DMSO solution and injected intraperitoneally. Vehicle mice were injected with a 4% DMSO solution.

Macrophage Preparation

Spleens were collected aseptically and placed in separate petri dishes containing cold (4° C.) Hanks Balanced Salt Solution without calcium and magnesium (Gibco BRL, Life Technologies Inc, Grand Island, N.Y.). Splenocytes were isolated by gentle mechanical disruption of the spleen with filtration through a nylon mesh (Spectrum, Laguna Hills, Calif.). The suspension was centrifuged for 7 minutes, at 500 g and 4° C. After resuspension, erythrocytes were lysed with an ammonium chloride solution for 4 minutes and the lysate was centrifuged as previously described. [23] The remaining pellet was resuspended in complete RPMI-1640 (Gibco BRL, Life Technologies Inc), containing 1% antibiotic/antimycotic solution and 10% heat-inactivated fetal bovine serum (Gibco BRL, Life Technologies Inc), counted and diluted to a final cell concentration of 5×10⁶ cells/ml and plated. After allowing macrophages to adhere for 2 hours, the remaining cells were suctioned off and macrophages were incubated with 10 ng/ml LPS 055:B5 (Sigma, St. Louis, Mo.) for PGE₂, IL-6 and TNFα determination, or (10 ng/ml LPS+100 U/ml recombinant mouse IFN-γ (Pharmingen, San Diego, Calif.) for NO₂⁻ determination. Cells were incubated at 37° C. in a 5% CO₂ atmosphere, and supernatants harvested at 24 hours and frozen at −70° C. for determination of prostaglandin, cytokine and NO production.

Immunoassay, Nitrite and Protein Determinations

Macrophage PGE₂, IL-6, and TNF-α production was assessed by means of specific enzyme-linked immunoassay (PGE₂ kit from Cayman Chemical, Ann Arbor, Mich., all other reagents from Pharmingen, San Diego, Calif.). NO₂⁻ was measured indirectly by determination of NO₂⁻ concentration using the Greiss reagent system (Promega, Madison, Wis.). The BCA protein assay (Pierce, Rockford, Ill.) was used to quantify protein and all measurements were normalized to protein content per well.

mRNA Isolation and COX-2 mRNA Determination

Isolated splenic macrophages were treated with lipopolysaccharide (LPS) (10 ng/ml) for six hours. Extraction of mRNA was with an RNA extraction kit (Qiagen, Inc.). Then, 1 μg of total mRNA was reverse transcribed in an RT mastermix (50 mM KCl, 10 mM Tris-HCl, 5 mM MgCl₂, 1 mM dNTP, 2.5 mM oligo d(T), 50 U RT, 20 U RNAse inhibitor, nuclease free water-all reagents Perkin Elmer) with an initial 10 minute incubation at room temperature and then 42° C. for 10 minutes. The 1 μl of cDNA product was for COX-2 (sequence: 5′-GCCCACCCCAAACACAGTGCAC-3′ as a sense primer, bases 259-280, and 5′-CTCGGAACCCCCAGTCCCTACTTG-3′ as an antisense primer, bases 594-571, primer pair A); and GAPDH (sequence: 5′-CAGGAGCGACCCCACTAA-3′ as a sense primer, and 5′-GGCATCGAAGGTGGAAGAGT-3′ as an anti-sense primer). The cDNA was run on a 1% agarose gel with 3 μl ethidium bromide 10 mg/ml solution), and bands visualized under UV transillumination and recorded in an image acquisition processing and analysis program (Eagle Eye 2, Stratagene).

Statistical Analysis

All cytokine results are reported as mean±SEM of 8 mice per group. Experimental results were evaluated for significance by ANOVA and where indicated by Newman-Kuehl's post-test. A value of p<0.05 was considered significant. For survival studies, Log-rank statistical analysis was used to compare all four groups studied (n=10-11 mice per group). A value of p<0.05 was considered significant.

Results

Body Weight, Spleen Weight, and Total Splenocyte Recovery

To report the effect of NS-398 treatment on general mouse morphology; body weight, spleen weight and total splenocyte number recovered per spleen were recorded. Table 1 shows the average body weight and total splenocytes per spleen (×10⁴) at seven days after injury on n=8 female BALB/c mice±SEM. As Table 1 indicates, at 7 days after trauma there was a significant difference in body weight in traumatized versus control mice (18.5±0.6 versus 20.2±0.9 g, respectively, p<0.001). Spleen weights were significantly increased in the trauma group, compared to controls (145±9 versus 93±6 mg, respectively, p<0.001) and the increased weight of spleens was reflected in part by an increased number of total splenocytes from 7.0±0.5×10⁴ to 10.1±10×10⁴ splenocytes per mouse in control versus injured mice (Table 1). Treatment with NS-398 did not significantly affect body weight, spleen weight and splenocyte number when compared to relative controls.

TABLE 1
Body weight, spleen weight, and splenocyte recovery
7 days after traumatic injury in balb/c mice

| | | | Total |
|---|---|---|---|
| | | Spleen | Splenocytes/ |
| Groups | Body wt g | wt mg | Mouse × 10⁶ |
| Control | 20.2 ± 0.3 | 92.9 ± 6.4 | 7.0 ± 0.5 |
| Control + NS-398 | 19.7 ± 0.2 | 87.3 ± 3.2 | 8.8 ± 0.6 |
| Trauma | 18.5 ± 0.2* | 144.8 ± 8.5* | 10.1 ± 1.0* |
| Trauma + NS-398 | 18.3 ± 0.2** | 125.3 ± 5.4** | 10.9 ± 0.8** |

*P < 0.05 for C v. T
**P < 0.05 for C v. TN

To evaluate the efficacy of NS-398 on plasma PGE₂ levels, mouse blood was collected at 6 hours after injury±treatment. Table 2 shows the mean values of pooled serum from n=6 mice per group. At 6 hours after trauma, plasma PGE₂ levels were increased from 38,535 pg/μl to 49,733 pg/μl (C vs T). After injured mice were treated with NS-398, plasma PGE₂ levels decreased to 29,902 pg/μl (TN) (Table 2).

TABLE 2
Plasma PGE₂ of mice at 6 h after traumatic injury

| | PGE₂ levels at 5 h |
|---|---|
| Groups | (pg/ml) |
| Control | 38,535 |
| Control + NS-398 | 35,961 |
| Trauma | 49,733 |
| Trauma + NS-398 | 29,902 |

Macrophage PGE₂, Cytokine and NO Patterns

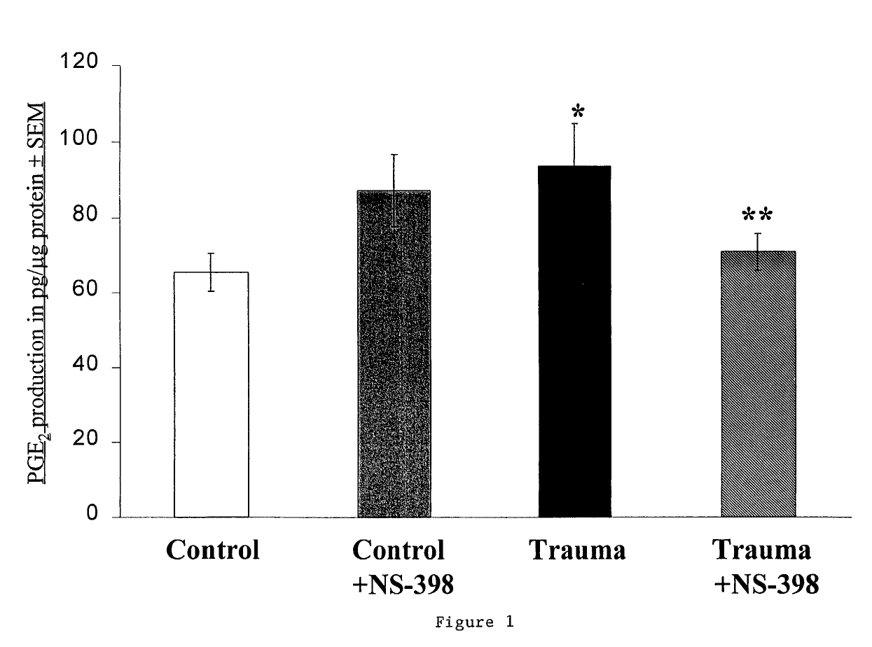

To evaluate the splenic macrophage production of measured mediators, supernatants were collected after 24 hours incubation. As FIG. 1 demonstrates, 7 days following injury, there was a significant increase in PGE₂ production to 100±11 pg/μg compared to 66±6 pg/μg from macrophages of control mice (T vs C). After in vivo treatment with NS-398 for 24 hours post-injury, there was a significant decrease in PGE₂ levels, to 71±5 pg/μg in trauma+NS-398 mice (p<0.03, T vs TN). Interestingly, there was also an increase in PGE₂ production from control mice treated with NS-398 to 87±10 pg/(g compared to placebo controls (C vs CN).

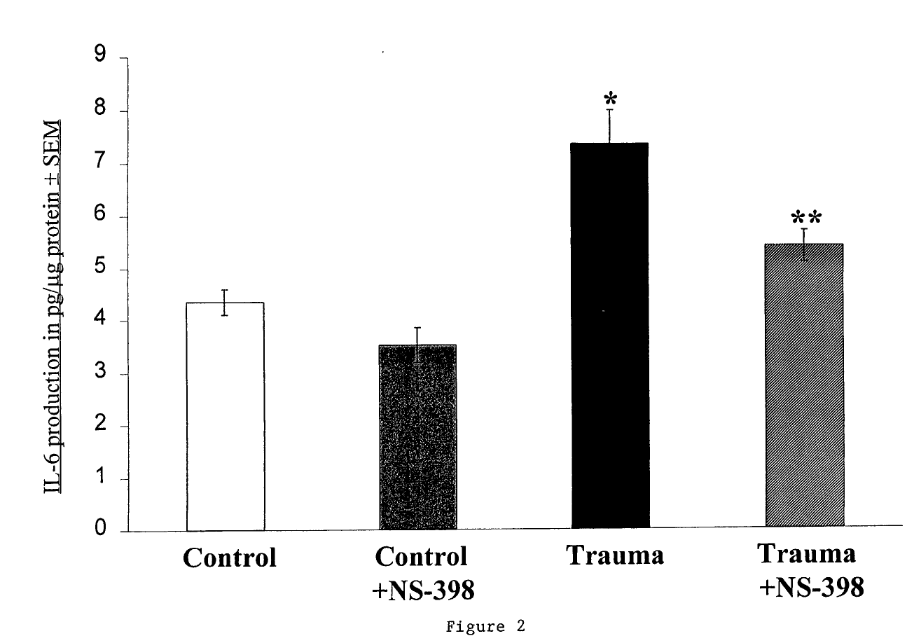

As FIG. 2 illustrates, IL-6 levels were significantly increased in the splenic macrophages of traumatized mice to 7.4±0.6 pg/μg, compared to only 4.4±0.3 pg/μg in macrophages of control mice (p<0.02, C vs T). NS-398 treatment, given to injured mice, decreased the level of IL-6 to 5.4±0.3 pg/μg, a significant decrease compared to traumatized placebo mice (p<0.02, T vs TN).

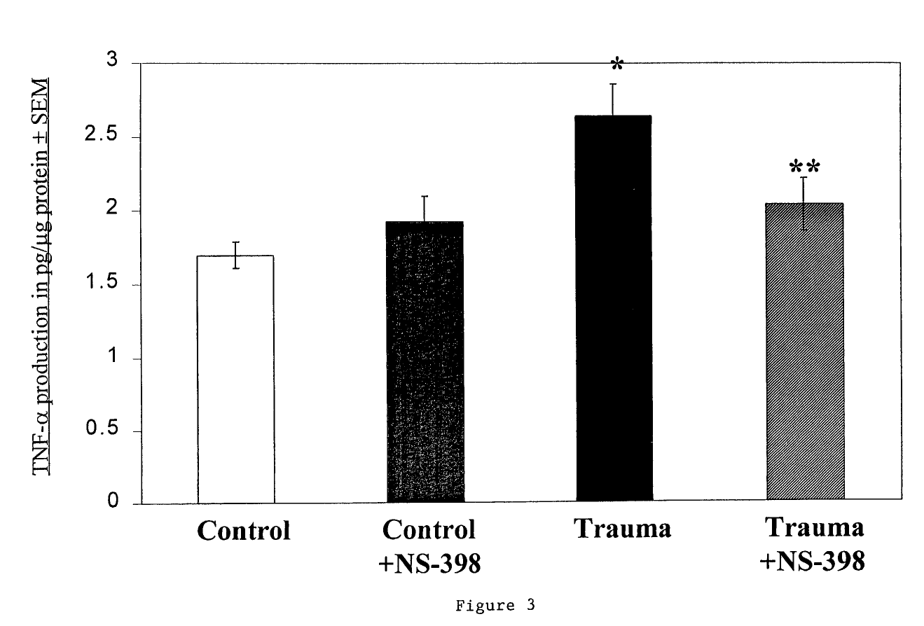

As FIG. 3 illustrates, at 7 days following trauma, there is a significant increase in TNF-α production, compared to controls (1.7±0.1 versus 2.7±0.2 pg/μl, C vs T) that decreases after treatment with NS-398 (2.7±0.2 versus 2.0±0.2 pg/μl, T vs TN, p<0.05).

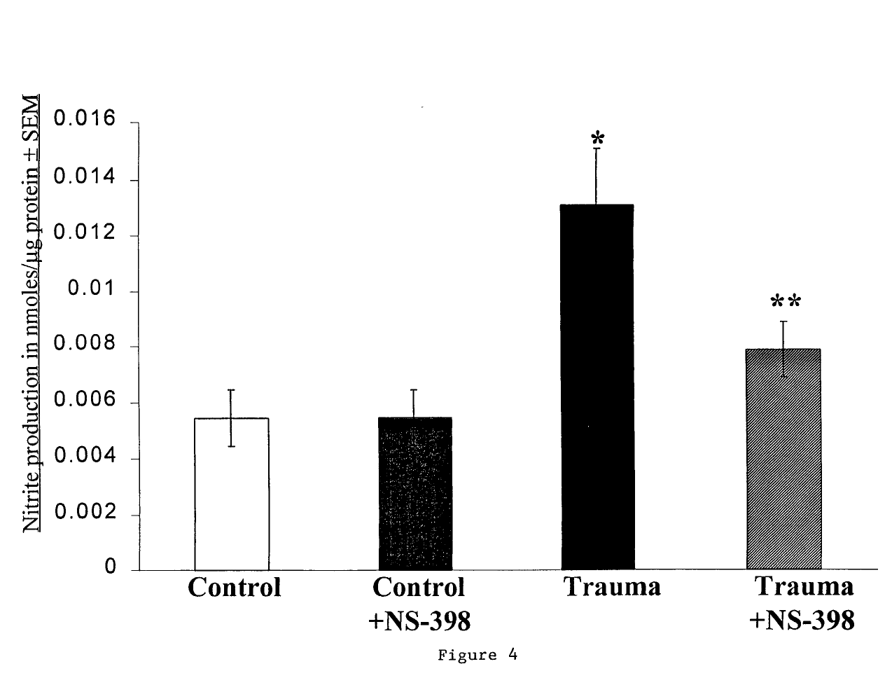

Significant increases in production of nitrite from LPS+IFN-γ stimulated splenic macrophages of traumatized mice to 0.013±0.006 nmoles/μg protein, compared to 0.005±0.003 nmoles/μg in controls p<0.01, C vs T) were also observed. The administration of NS-398 to injured mice significantly decreased nitrite levels to 0.007±0.004 nmoles/μg compared to traumatized mice (p<0.02), FIG. 4.

COX-2 mRNA

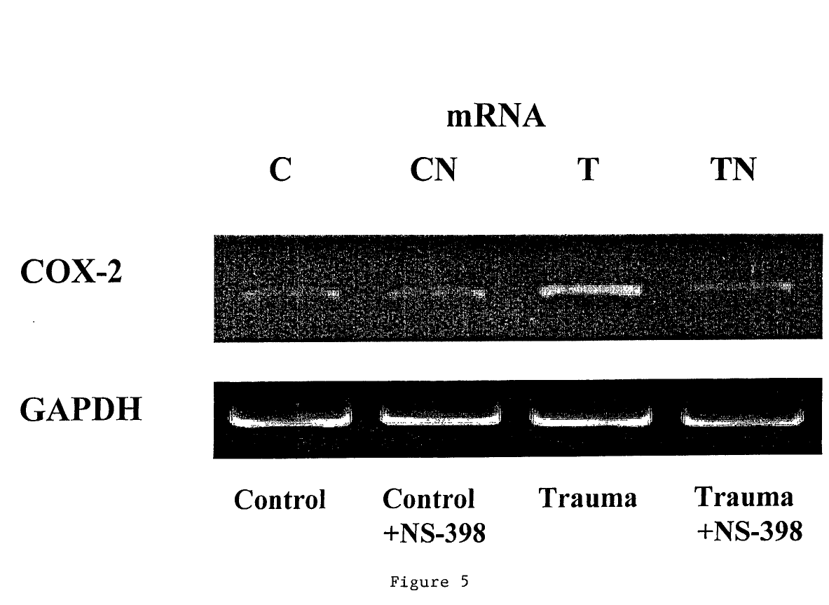

To evaluate the relative expression of COX-2 mRNA, splenic macrophages were treated as previously described and harvested by RT-PCR. FIG. 5 demonstrates the relative levels of inducible COX-2 mRNA in the four experimental groups compared to the constitutively expressed levels of GAPDH. Whereas stimulated splenic macrophages from control mice exhibited a lower level of COX-2 mRNA expression, stimulated splenic macrophages from traumatized mice showed a marked induction of COX-2 mRNA. Treatment with NS-398 attenuated the levels of COX-2 mRNA to below control levels. In contrast, the levels of GAPDH run from the same macrophages show no difference in expression of GAPDH mRNA among the four groups examined (FIG. 5).

Survival Patterns of Mice

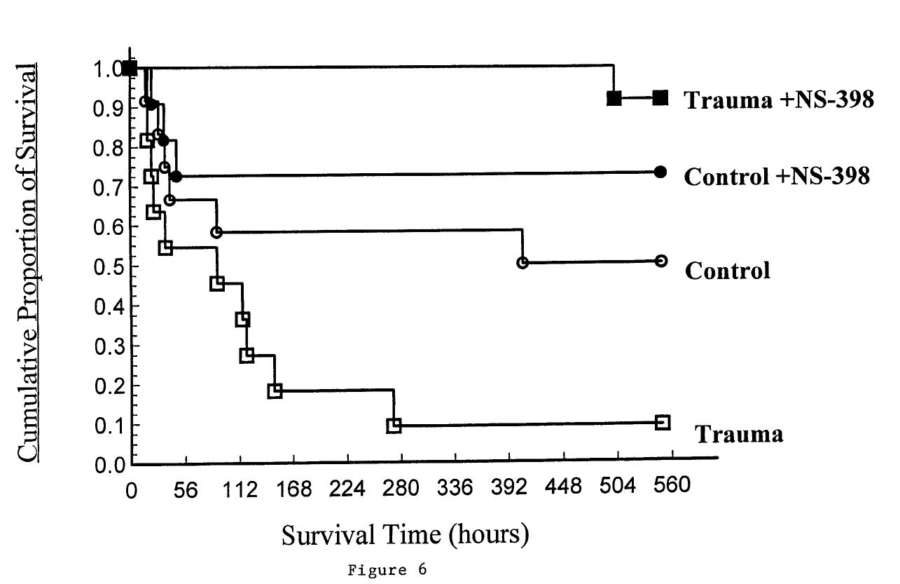

To assess the biological relevance of NS-398, mice were treated as above and on day 7 after trauma, given a septic challenge via cecal ligation and puncture (CLP). Mice were then followed for survival over 24 days at which time, remaining mice were euthanized. As FIG. 6 illustrates, after septic challenge, uninjured mice reached a survival of 50% after 24 days. Injured mice showed a survival of 10% at 24 days after CLP. In contrast, traumatized mice treated with NS-398 had a significant survival advantage of 92% after 24 days (p<0.001, T vs TN). Control mice treated with NS-398 also exhibited a survival advantage compared to control placebo mice of 66%, although this was not statistically significant when compared to control.

The foregoing results reflect a significant survival advantage in traumatized mice treated within 24 hours with the selective COX-2 inhibitor, NS-398 compared with traumatized mice not administered NS-398. These data therefore identifies an important role for modulation of COX-2 in the process of immune dysfunction following severe injury.

Not only was it found that NS-398 treatment resulted in decreased levels of PGE₂, but COX-2 mRNA expression, as well as IL-6, TNF-α and NO production at 7 days after injury were also decreased.

In summary, results indicate that in vivo treatment of injured mice with NS-398 within the first 24 hours significantly decreases PGE₂ levels and COX-2 mRNA levels. In addition, NS-398 normalizes critical post-injury pro-inflammatory mediators that are associated with decreased immunity. Most importantly, NS-398 given to injured mice in vivo, improves their survival after a septic challenge.

### EXAMPLE II

### Blocking Prostaglandin-E2 After Trauma is Associated with Decreased COX-2 mRNA Expression and Improved Survival

Balb/c mice (n=8/group) were given 3 intraperitoneal injections of 10 mg/kg NS398; immediately following injury, at 12 and 24 hours after sham injury or trauma (femur fracture and 40% blood volume hemorrhage). Sham+placebo (C); sham+NS398 (CN); trauma+placebo (T); or trauma+NS398 (TN) groups were studied. On day 7 after injury, mice were sacrificed and splenic macrophages evaluated for cytokine production and expression of COX-2 mRNA. In a separate study, mice (n=10-12/group) were treated in an identical fashion. After 7 days, cecal ligation and puncture was performed and animals followed for survival.

TABLE 3

| | | | IL-6 | | NO |
|---|---|---|---|---|---|
| | | | (pg/μg | | (nmol/ |
| Group | Survival | PGE₂ | protein) | TNF-α | μgprot) |
| C | 50% | 66 ± 14 | 4.4 ± 0.7 | 1.7 ± 0.3 | 5 ± 3 |
| T | 10% | 94 ± 32ˆ | 7.4 ± 1.8ˆ | 2.6 ± 0.6ˆ | 13 ± 6ˆ |
| T + NS | 92%# | 71 ± 14* | 5.4 ± 0.9* | 2.0 ± 0.5* | 8 ± 4* |

Data=mean±SEM; #p<0.01 by log-rank test (TvsTN); *p<0.05 (TvsTN) and ˆ p<0.05 (CvsT) by student's t-test. Control+NS398 data similar to Control data.

As indicated in Table 3, traumatized mice treated with NS398(TN) had a significant improvement in survival compared to trauma mice alone (T). Splenic macrophages from traumatized mice (T) had significant increase in PGE₂, IL-6, TNF-α and NO versus control mice (C) and marked increases in COX-2 mRNA. Treatment of injured mice with NS398 caused significant decreases in PGE₂, IL-6, TNF-α production and COX-2 mRNA (FIG. 5). Thus, NS398 not only suppresses PGE₂ through enzymatic pathways, but likely also acts via transcriptional pathways to alter mRNA expression and provide a survival advantage to the host. Importantly, only 3 doses of NS398 given within 24 hours after injury significantly improved survival to a later septic challenge.

### EXAMPLE III

### NS-398 Treatment After Trauma Modifies NF-κB Activation and Improves Survival

### Materials and Methods

Animals, Experimental Protocol, NS-398 preparation were as described in Example I. Macrophages were prepared as described initially under “Macrophage Preparation ” in Example I. After lysis of erythrocytes with ammonium chloride solution and centrifugation as described in Example I, macrophages were further prepared by resuspending the pellet in RPMI-1640 (Gibco BRL, Life Technologies Inc) containing 2 mmol/L L-glutamine, 100 U/mL penicillin, 100 μg/mL streptomycin, 0.25 μg/mL amphotericin B, and 10% heat-inactivated fetal calf serum (Gibco BRL, Life Technologies Inc), counted via hemocytometer and diluted to a final concentration of 5×10⁶ cells/mL, and plated in 96 well plates in a volume of 0.2 mL per well. After allowing macrophages to adhere for 2 hours, the remaining cells were suctioned off and the macrophages were cultured in the +/−1 ng LPS 055:B5 (Sigma, St. Louis, Mo.) per mL at 37° C. in 5% carbon dioxide incubators. Supernatants were harvested at 24 hours and stored at −70° C. until analysis for batch enzyme-linked immunosorbent assay processing to determine prostaglandin and cytokine production.

Immunoassay, Nitrite and Protein Determinations

Macrophage PGE₂, leukotriene-B4 (LTB₄), IL-6, and TNF-α production was assessed by ELISA (PGE₂ and LTB₄ kit from Cayman Chemical, Ann Arbor, Mich., all other reagents from Pharmingen, San Diego, Calif.). NO was measured indirectly by NO₂⁻ concentration using the Greiss reagent system (Promega, Madison, Wis). Protein was quantified using the BCA protein reagent (Pierce, Rockford, Ill.) and all measurements were normalized to protein content per well.

mRNA COX-2 Sample Preparation

Isolated splenic macrophages were treated with LPS (10 ng/mL) for 6 hours. Extraction of mRNA was done with an RNA extraction kit (Qiagen, Inc.). Total mRNA (1 μg) was reverse transcribed in an RT mastermix (50 mM KCl, 10 mM Tris-HCl, 5 mM MgCl₂, 1 mM dNTP, 2.5 mM oligo d(T), 50 U RT, 20 U RNAse inhibitor, nuclease free water), (all reagents Perkin Elmer) with an initial 10 minute room temperature incubation and then 42° C. for 10 minutes. The 1 μl of cDNA product was for COX-1 (sequence: 5′AGTCGAAGGAGTCTCTCGCT CTGG-3′ as a sense primer, bases 40-63, and 5′-CAGGAAATGGGTGAACGAGGGGCT-3′ as an antisense primer, bases 318-295), COX-2 (sequence: 5′-GCCCACCCCAAACACAGTGCAC-3′ as a sense primer, bases 259-280, and 5′-CTCGGAACCCCCAGTCCCTACTTG-3′ as an antisense primer, bases 594-571, primer pair A), and GAPDH (sequence: 5′-CAGGAGCGACCCCACTAA-3′ as a sense primer, and 5′-GGCATCGAAGGTGGAAGAGT-3′ as an anti-sense primer). cDNA was run on a 1% agarose gel with 3 μL ethidium bromide (10 mg/mL solution) and bands visualized under UV transillumination and recorded in an image acquisition processing and analysis program (Eagle Eye 2, Stratagene). mRNA was quantified by RT-PCR and normalized to GAPDH.

Electrophoretic Mobility Shift Assay (EMSA)

NF-κB gel shift oligonucleotide 5′ AGT TGA GGG GAC TTT CCC AGG C 3′ (Santa Cruz Laboratories, Santa Cruz, Calif.) was end labeled with (³²P)γATP using polynucleotide kinase T4 (Gibco BRL, Grand Island, N.Y.). End labeled probe was purified from unincorporated (³²P)-γATP using a purification column (Bio-Rad Laboratories) and recovered in tris-EDTA buffer. Samples consisted of adherent macrophages collected after 45 minutes stimulation with 10 ng/mL LPS. Adherent cells were scraped into 1 mL of cold HANKS balanced salt solution. The cell suspension was pelleted after centrifugation in a microcentrifuge for 10 seconds and resuspended in 400 μl cold Buffer A (10 mM HEPES-KOH pH 7.9 at 4° C., 1.5 mM MgCl₂, 10 mM KCl, 0.5 mM dithiothreitol, 0.2 mM PMSF) by agitation of the tube. The cells were allowed to swell on ice for 10 minutes, and then vortexed for 10 seconds. Samples were centrifuged for 10 seconds and the supernatant fraction was saved at −70° C. for IκB analysis. The pellet was resuspended in 20-100 μl of cold buffer C (20 mM HEPES-KOH pH 7.9, 25% glycerol, 420 mM NaCl, 1.5 mM MgCl₂. 0.2 mM EDTA, 0.5 mM dithiothreitol 0.2 mM PMSF) and incubated on ice for 20 minutes. Samples were microcentrifuged for 2 minutes and supernatants collected and stored at −70° C. for batch analysis. Labeled probe was added to samples using standard protocols (standardized by protein), and then incubated for 20 min. Samples were subjected to electrophoretic separation on a non denaturing 5% poly-acrylamide gel at 30 mA using Tris borate EDTA for 45 minutes and analyzed by exposure to radiographic film (Kodak, Rochester, N.Y.).

Western Blot

Cytoplasmic fractions were collected after splenic macrophages were stimulated (as described above) and then boiled in equal volumes of loading buffer (125 mM Tris-HCl, pH 6.8, 4% sodium dodecyl sulfate, 20% glycerol and 10% 2-mercaptoethanol) for 3 minutes and were then separated by electrophoresis on a tris-glycine gradient gel. The proteins were then transferred to nitrocellulose membranes (Xcell II Blot Module; Novex). The membranes were blocked with 10% non-fat dried milk in Tris-buffered saline (TBS), pH 7.6, containing 0.05% tween-20 (TTBS) for 1 hour and then incubated with a polyclonal rabbit anti-mouse antibody to IκB-α (Santa Cruz Laboratories) for 45 minutes. After washing twice in TTMS, the blots were incubated with a peroxidase-conjugated goat anti-rabbit IgG secondary antibody for 15 minutes. The blots were washed in TTBS for 5 minutes three times, then in TBS for 5 minutes, incubated in enhanced chemiluminescence reagents (ECL, Amersham Life Sciences, Buckingham, England) and exposed on radiographic film (Kodak, Rochester, N.Y.).

Statistical Analysis

All cytokine results are reported as mean±SEM of 8 mice per group. The ANOVA was used to determine the significance of the differences between means of traumatized±treatment versus control±treatment groups. A P value of <0.05 was considered significant. For survival studies, Kaplan-Meier (Log-rank) statistical analysis was used to compare all four groups studied. A P value of <0.05 was considered significant.

Results

Body Weight and Total Splenocyte Recovery

Body weight and splenocyte recovery after injury±NS-398 were recorded. At 7 days after trauma there was no significant difference in body weight observed in the control versus trauma or control versus trauma+NS-398 mice (Table 4). Furthermore, there were no differences in splenocyte counts for the control versus trauma±NS-398 mice.

TABLE 4
Body Weight and Splenocyte Counts 7 Days After Trauma

| | | Total Splenoctyes/ |
|---|---|---|
| Group | Body Wt. (g) | mouse (× 10⁶) |
| Control | 17.7 ± 0.2 | 7.0 ± 1.3 |
| Control + NS-398 | 18.3 ± 0.3 | 8.8 ± 1.6 |
| Trauma | 16.6 ± 0.2 | 10.1 ± 2.8 |
| Trauma + NS-398 | 16.7 ± 0.7 | 10.9 ± 2.3 |

Plasma PGE₂ Levels

Plasma PGE₂ levels were determined in separate experiments by sacrificing mice at 1 day and at 7 days after treatment with NS-398. There was an increase in PGE₂ levels at 1 and 7 days after traumatic injury compared with uninjured controls. PGE₂ levels were attenuated in both control and injured mice by treatment with NS-398 to below control levels (Table 5).

TABLE 5
Plasma PGE₂ Levels at 1 and 7 Days After Trauma

| Group | Day 1 (pg/ml) | Day 7 (pg/ml) |
|---|---|---|
| Control | 28,511 | 12,978 |
| Control + NS-398 | 24,652 | 7,244 |
| Trauma | 37,916 | 14,574 |
| Trauma + NS-398 | 26,370 | 4,354 |

Splenic Macrophage PGE₂ and LTB₄ Patterns

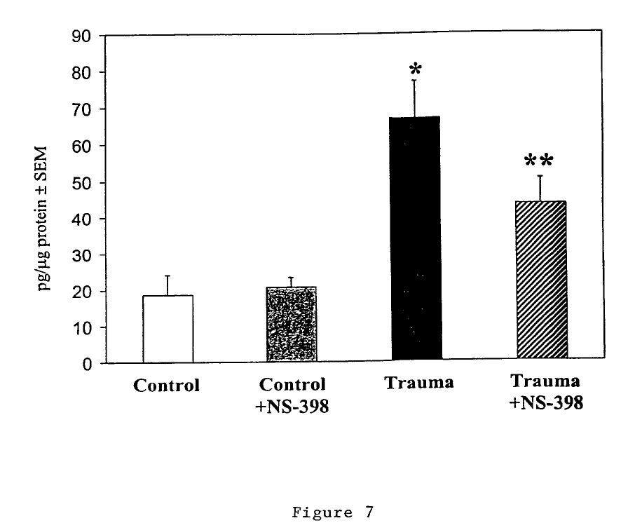

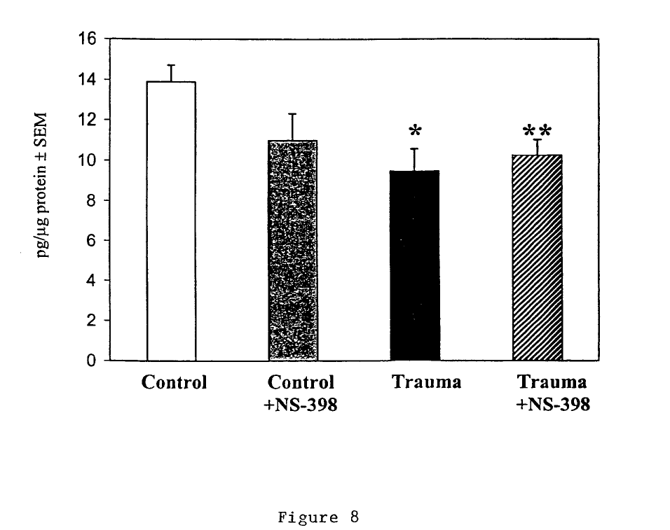

As depicted in FIG. 7, 7 days after trauma, PGE₂ levels are significantly increased. After treatment with NS-398, splenocytes from traumatized mice produce significantly less PGE₂ (P<0.05) and approached levels observed in controls (FIG. 7). After trauma, there was a significant decrease in LTB₄ levels compared to macrophages of control mice (P<0.03). NS-398 given after injury, however, did not restore LTB₄ levels to those seen in control (FIG. 8).

Splenic Macrophage TNF-α, NO and IL-6 Levels

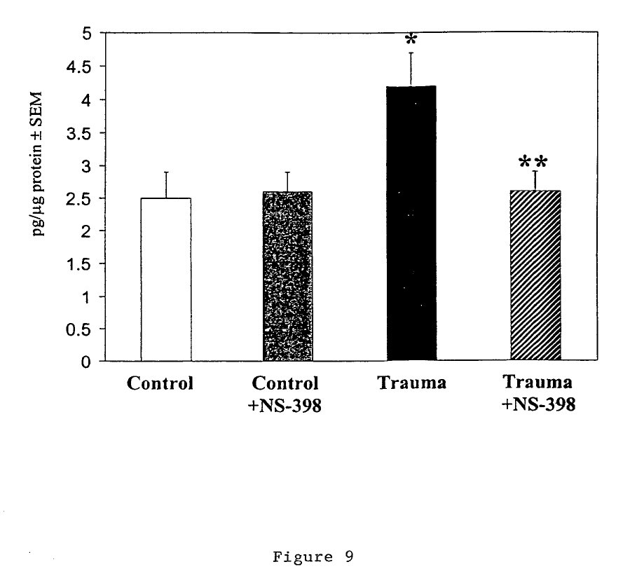

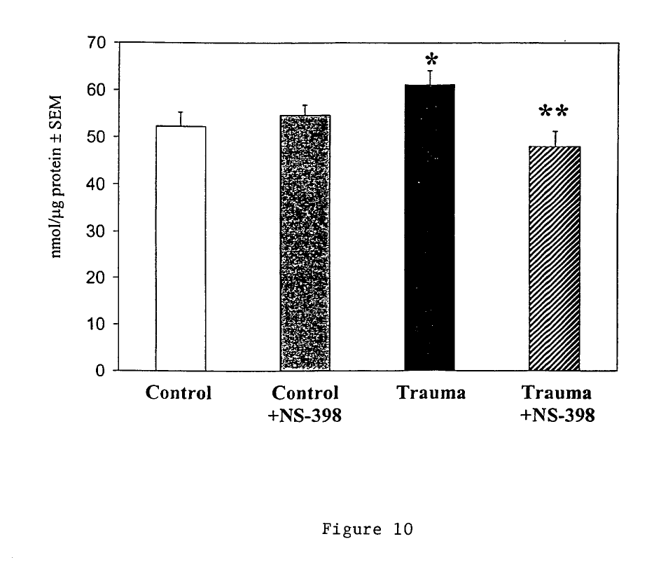

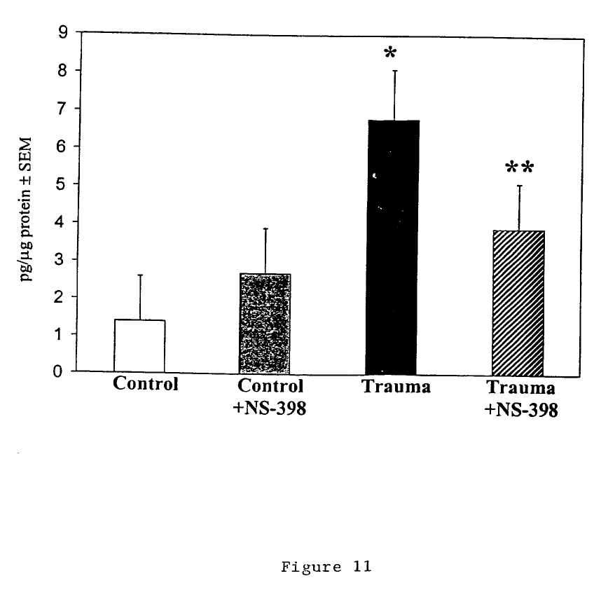

TNF-α, NO and IL-6 levels were significantly increased after trauma from stimulated splenic macrophages when compared to production from control groups (P<0.05). Treatment with NS-398 significantly attenuated production of these mediators compared to untreated injured mice (P<0.05) (FIGS. 9-11).

COX-2 mRNA and NF-κB/IκB Results

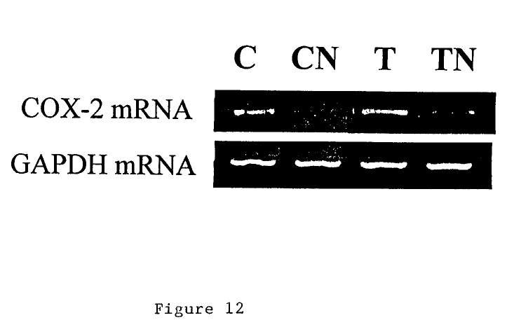

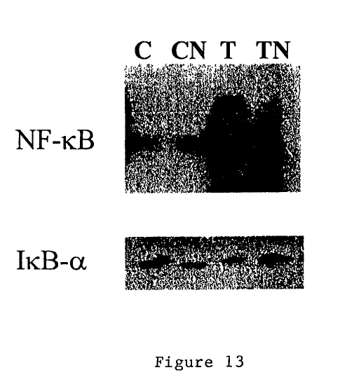

As depicted in FIG. 12, trauma induced COX-2 mRNA levels and NS-398 decreased this induction. After injury, cystolic levels of IκB decreased and NF-κB increased. NS-398 treatment after trauma, resulted in an increase in cytosolic IκB and a decrease in NF-κB activation (FIG. 13). Thus, it appears that trauma signaled the dissociation of the IκB/NF-κB complex which caused degradation of cytosolic IκB-α (decreased levels after trauma), and allowed NF-κB to migrate intra-nuclearly to activate transcription. NS-398 treatment appears to have prevented this dissociation of the IκB/NF-κB complex and transcriptional activation, resulting in higher levels of IκB and lower levels of NF-κB, similar to that seen in macrophages from control mice.

Survival Patterns of Mice after Septic Challenge

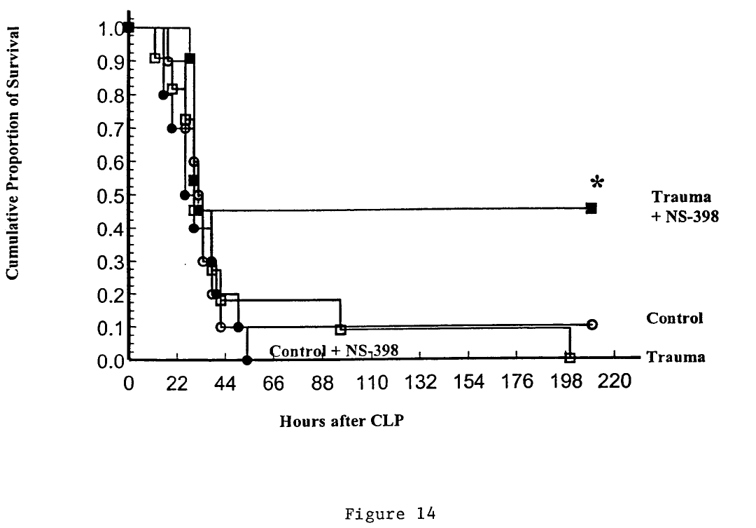

To evaluate the biological relevance of NS-398, mice were randomized into the four treatment groups. After 7 days, all mice were subjected to cecal ligation and puncture (CLP) and followed for survival over 10 days, when remaining mice were euthanized. FIG. 14, illustrates the Kaplan-Meier survival plots and demonstrates that trauma and control+NS-398 mice died shortly after CLP. Only 9% of control mice survived after 200 hours compared to a significant increase in survival of trauma+NS-398 treated mice in which 45% survived.

The foregoing results demonstrate that injured mice treated with NS-398 over 7 days after injury have a significant survival advantage when compared with non-treated mice. In Example I, the effect of administering NS-398 only during the initial 24 hours after injury was examined; during which a surge in prostaglandins is initiated, and which was associated with a significant survival advantage when compared to vehicle-treated injured mice.

These results confirm that pro-inflammatory mediator responses at 7 days after trauma are similar to those observed with a shorter (24-hour) course of treatment. It was found that 7 days of NS-398 treatment normalized PGE₂, TNF-α, IL-6 and NO levels similar to levels produced by macrophages from control mice.

Continuous administration of NS-398 over 7 days did not result in higher survival rates than in Example I where mice were treated for only the first 24 hours after injury.

In summary, NS-398 given over 7 days after trauma, significantly attenuates macrophage production of PGE₂, TNF-α, IL-6 and NO, but does not affect production of LTB₄. In addition, treatment with a COX-2 inhibitor modulates macrophage NF-κB activation. Significant survival advantage is confirmed in injured mice treated with a selective COX-2 inhibitor.

### EXAMPLE IV

### NS398 Treatment After Trauma Attenuates Inflammatory Response and Increases Survival

Prostaglandin-E₂ (PGE₂) production after trauma contributes to immune alterations that may increase susceptibility to infectious complications. This study evaluated the effect of NS398 given over 7 days on pro-inflammatory cytokines and survival after a septic challenge.

BALB/C mice (n=8/group) were given 10mg/kg NS398 intraperitoneally BID for 7 days, starting immediately after sham injury or trauma (femur fracture+40% hemorrhage). Four groups, sham+placebo (C); sham+NS398 (CN); trauma+placebo (T); or trauma+NS398 (TN) were studied. On day 7 after trauma, mice were sacrificed and splenic macrophages evaluated for PGE₂, TNF-α and NO production (via Student's t-test). In a separate study, mice (n=10-11/group) were traumatized and given NS398 for 7 days. On day 7, cecal ligation and puncture was performed, and mice evaluated for survival over 3 weeks (via Log-rank test).

TABLE 6

| Groups | Survival | NO (nmol/μg protein) | TNF-α (pg/μg protein) |
|---|---|---|---|
| C | 9% | 52.3 ± 3.1 | 2.5 ± 0.4 |
| C + NS398 | 0% | 54.8 ± 2.1 | 2.6 ± 0.3 |
| T | 0% | 61.2 ± 3.1ˆ | 4.2 ± 0.5ˆ |
| T + NS398 | 45%* | 48.0 ± 3.3^{#} | 2.6 ± 0.3^{#} |

(Data = mean ± SEM;
*p < 0.02 by Log rank (Mantel-Cox) test (T vs TN),
^{#}p < 0.05 by Student's t-test (T vs TN)/ˆ (C vs T).

NS398 treatment of injured mice (TN) decreased PGE₂ production compared to trauma+placebo (T) (3.9±0.3 vs 3.1±0.4pg/μg protein) and resulted in a significant survival advantage for traumatized mice given NS398. Additionally, the increased production of TNF-α and NO from splenic macrophages in traumatized mice was normalized after treatment with NS398 (Table 6).

These data indicate that daily treatment with a specific COX-2 inhibitor not only suppresses PGE₂, but normalizes key pro-inflammatory cytokines after trauma. These findings correlate with significantly increased survival in injured mice treated with NS398 and given a subsequent septic challenge, and indicates that COX-2 inhibitors can play an important role in modulating the inflammatory response and improving survival after trauma.

### EXAMPLE V

### PGE2 Receptors EP2 and EP4 Are Downregulated in Human Mononuclear Cells After Injury

### Materials and Methods

Subjects

The study included 10 patients sustaining either long-bone fracture or ≧15% total body surface burn and 10 control subjects (protocol approved by the Weill Medical College of Cornell University Internal Research Board). Blood samples were obtained within 12 hours of fracture or burn injury. Immune compromised patients including those with cancer, collagen-vascular diseases, HIV or any recent history of steroid or NSAID drug use were excluded from this study. Each patient sample was run simultaneously with a control sample.

Peripheral Blood Mononuclear Cell Isolation and Stimulation

Peripheral venous blood samples were collected into two heparin-EDTA tubes and placed on ice. 6 mL of Histopaque (Sigma Chemical Co., St. Louis, Mo.) was layered into a conical 15 mL tube and 6 mL of undiluted whole blood was gently layered over the top. Tubes were then centrifuged at 1000 rpm for 30 minutes at room temperature. A sterile pasteur pipet was used to gently collect the layer of PBMC's which were then transferred to a new 15 mL tube. Samples were washed twice with 10 mL Ca⁺² and Mg⁺²-free Hanks Buffered Salt Solution (Gibco BRL-Life Technologies, Grand Island, N.Y.) and centrifuged at 1100 rpm for 10 minutes. The supernatant was discarded and the pellet resuspended in complete RPMI-1640 (Gibco BRL-Life Technologies, Grand Island, N.Y.) with 10% heat inactivated fetal bovine serum (Gibco BRL-Life Technologies, Grand Island, N.Y.) and 1% antibiotic/antimycotic solution. Cells were then adjusted to a final concentration of 1×10⁶ cells/mL and plated. Cells were incubated at 37° C. in a 5% CO₂ atmosphere with supernatants harvested at 24 hours and frozen at −70° C. for determination of prostaglandin and cytokine production. Cell viability was confirmed via Trypan Blue exclusion and showed >95% cell viability.

PGE₂, LTB₄ and TNF-α Immunoassay Measurements

Supernatants from stimulated PBMC's were assessed by specific enzyme-linked immunoassay (PGE₂ and LTB₄ kits from Cayman Chemical, Ann Arbor, Mich.; all other reagents from Pharmingen, San Diego, Calif.). PGE₂ (Sigma Chemical Co., St. Louis, Mo.) used in co-culture experiments was dissolved in 0.1% DMSO hand all corresponding controls were co-cultured with 0.1% DMSO vehicle.

RNA Preparation and RT-PCR

Isolated PBMC's from various groups were incubated for 18 hours at which time all groups were treated with 10 ng/mL lipopolysaccharaide (LPS) 055:B5 (Sigma, St. Louis, Mo.) for 6 hours RT-PCR samples were run in a blinded fashion. Extraction of mRNA was with an RNA extraction kit (Qiagen). Total mRNA (1 μg) was reverse transcribed in an RT mastermix (50 mM KCl, 10 mM tris-HCl, 5 mM MgCl₂, 1 mM dNTP, 2.5 mM oligo d(T), 50 units of RT, 20 units of RNAse inhibitor, and nuclease free water; all reagents from Perkin-Elmer, Foster City, Calif.) with an initial 10 minute incubation at room temperature and then 42° C. for 10 minutes. The 1 μL of cDNA product was for EP1 (sense, CCA CCA CCT TCC TTC TGT TCG; anti-sense, GGT GGG CTG GCT TAG TCG TTG); EP2 (sense, CTT ACC TGC AGC TGT ACG; anti-sense, GAT GGC AAA GAC CCA AGG); EP3 (sense, CGC GTC AAC CAC TCC TAC ACA; anti-sense, GCA GAC CGA CAG CAC GCA CAT); EP4 (sense, GGT CAT CTT ACT CAT TGC CAC C; anti-sense, AGA TGA AGG AGC GAG AGT GG); CD14 (sense, ACT CCC TCA ATC TGT TCG CTG; anti-sense, CTG AAG CCA AGG CAG TTT GAG TCC); COX-2 (sequence: sense GCC CAC CCC AAA CAC AGT GCA C, bases 259-280; antisense CTC GGA ACC CCC AGT CCC TAC TTG, bases 594-571); and GAPDH (sequence: sense CAG GAG CGA CCC CAC TAA; anti-GGC ATC GAA GGT GGA AGA GT). The cDNA was run on a 1% agarose gel with 3 μL of ethidium bromide (10 mg/mL solution) and bands were visualized under UV transillumination and recorded in an image acquisition processing and analysis program (Eagle Eye 2, Stratagene).

NS-398 Preparation

NS-398 (N-[2-(cyclohexyloxy)-4-nitrophenyl]-methanesulfonamide) was obtained from Cayman Chemical (Ann Arbor, Mich.) and prepared in DMSO at a concentration of 1 mM. Serial dilutions were made to co-culture cells with a final concentration of 10 mM NS-398 in a 0.1% DMSO. Control and trauma groups were co-cultured with 0.1% DMSO vehicle for comparisons with NS-398 treated groups.

Statistics

All values represent the mean±S.E.M. for n=10 patients per group. Analysis of variance was performed to evaluate differences in PGE₂, LTB₄ and TNF-α production. Statistical differences were obtained by two-tailed Newman-Kuehl's post-test for compared samples and a P values of <0.05 were considered significant.

Results

Patients

Patients averaged 40.8 years of age (range 20-66) and included 6 males and 4 females. Four patients had suffered fractures and 6 patients had suffered burn injury (average 22% burn, range 15-28%). Control subjects were volunteers averaging 28.9 years of age (range 24-42) and included 4 females and 8 males.

Eicosanoid and Cytokine Production by PBMC's

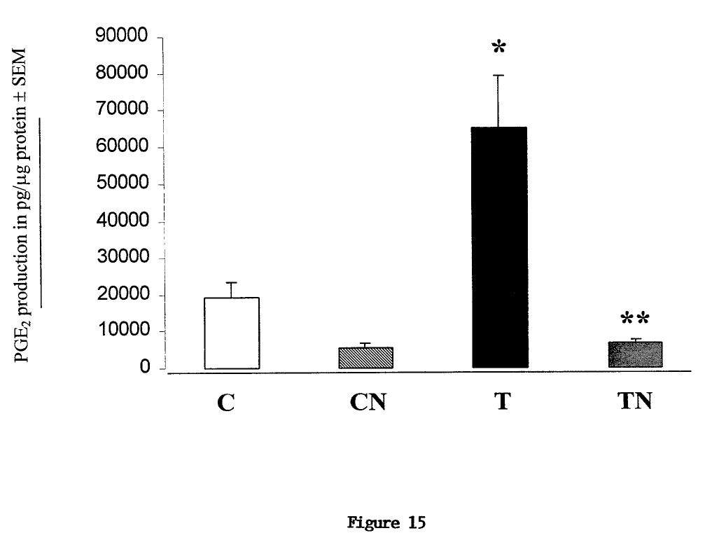

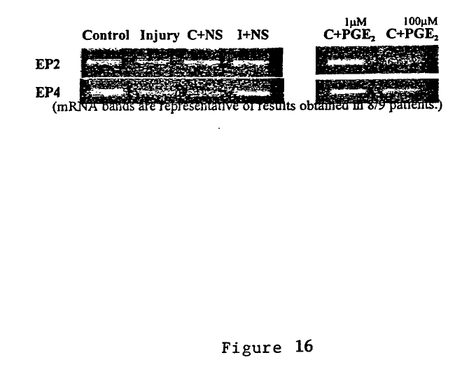

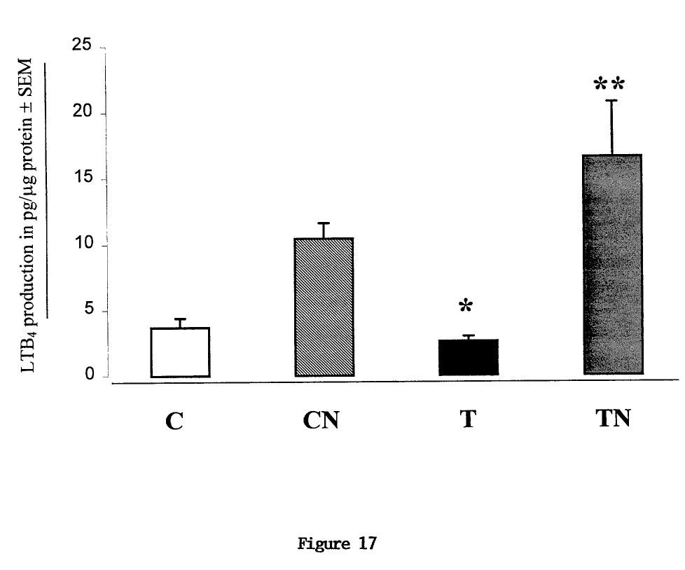

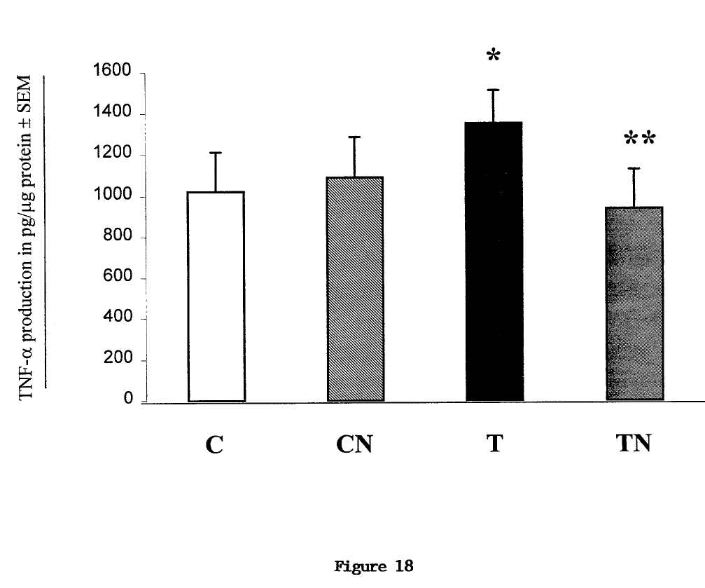

FIG. 15 demonstrates that after injury (I), there was a significant increase in PGE₂ production (65,100×14,200 vs 19,400±4200 pg/ml, I vs C, P<0.0001) when compared to controls (C vs T) and that co-culture with 10μM NS-398 significantly reduced the production of PGE₂ (P<0.001, T vs. TN). FIG. 17 demonstrates the production of LTB₄ from stimulated PBMC's and shows that although there is no significant decrease in LTB₄ production in peripheral cells of injured patients, there is a significant increase in the production of LTB₄ after co-culture with NS-398 in trauma cells (P<0.004, T vs TN) as well as in control cells (P<0.05, C vs CN). With regard to TNF-α production, FIG. 18 shows that TNF-α production was increased after injury (P<0.05, C vs T) and that co-culture with NS-398 significantly decreased production (P<0.05, T vs TN).

EP Receptor Identification in PBMC's

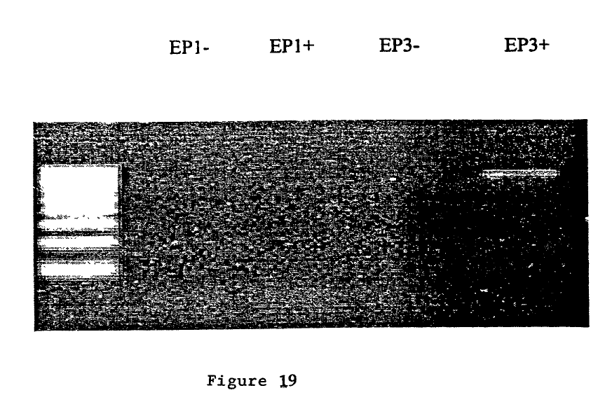

FIG. 19 shows the presence of EP1 and EP3 in a lymphoma cell line and in a renal cell carcinoma cell line, however, EP1 and EP3 receptor expression was undetectable in both simulated and unstimulated PBMC samples.

EP receptor expression in PBMC's and relation to PGE₂

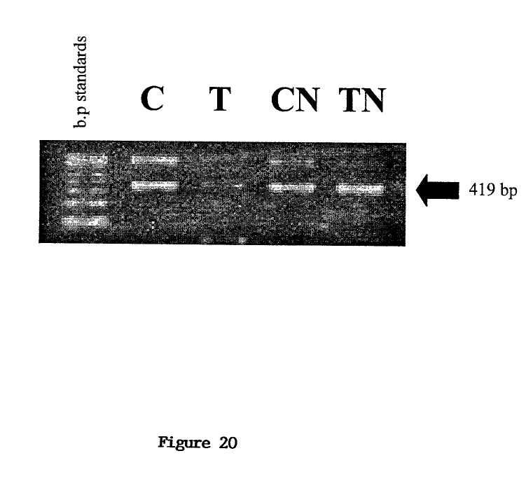

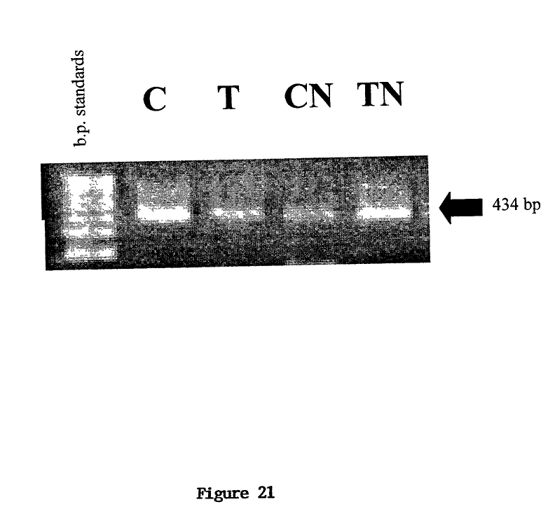

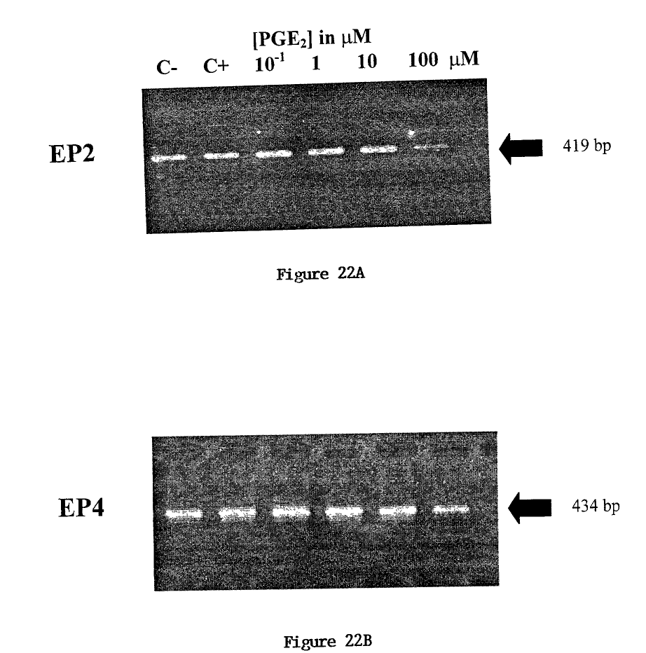

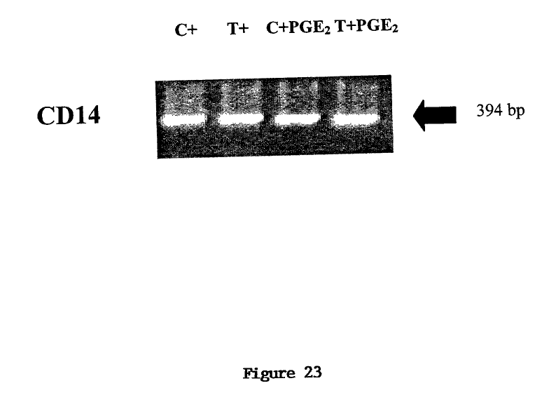

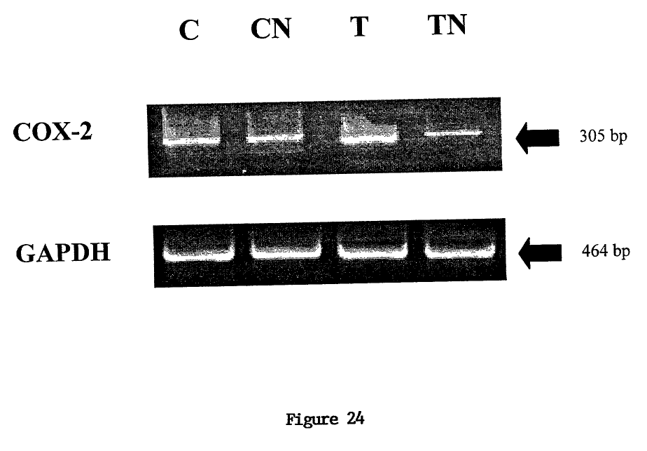

FIG. 20 shows that after injury, there is a decrease in EP2 receptor expression that returns to control levels after co-incubation with NS-398. FIG. 21 shows decreased expression of the EP4 receptor in PBMC's after injury reversed by co-incubation with NS-398.

FIGS. 21A and 21B demonstrate the effect of increasing doses of exogenous PGE₂ on the expression of the EP2 and EP4 receptors from control cells. Stimulation with LPS does not cause further induction of EP2 and EP4. As the dose of exogenous PGE₂ increases, there is no change in receptor expression of either EP2 or EP4 until 100 μM of PGE₂ is added. At this concentration, both EP2 and EP3 receptor expression is attenuated to levels below those seen in control samples.

Expression of CD14 Markers from PBMC's

The expression of CD14 markers from mononuclear cells was also evaluated in control and injured samples stimulated with LPS. All of the samples showed similar expression of the CD14 marker.

Expression of COX-2 from PBMC's

To further evaluate the expression of COX-2, samples of LPS stimulated PBMC's were evaluated from control and injured subjects as well as samples co-incubated with 10 μM NS-398. After injury, there is a marked induction of COX-2 in the cells from injured patients. Furthermore, NS-398 reduced COX-2 expression to levels similar to that expressed by control cells.

These results demonstrate that there is injury-mediated downregulation of the human PGE2 receptors, EP2 and EP4.

Whether PGE₂ was at least partially responsible for the downregulation of the EP2 and EP4 receptors was evaluated by co-incubating PBMC's with exogenous PGE₂ (10⁻⁴ μM up to 100 μM). No attenuation in EP2 and EP4 receptors was noted up to 100 μM PGE₂. Although a high dose of PGE₂, this dose was not toxic to the cells studies as demonstrated by up regulation of the CD14 receptors in control and PGE₂-treated samples stimulated with LPS.

To confirm that injury was not down regulating all circulating mononuclear cell surface receptors, the expression of the CD14 receptor, which is ubiquitous on the surface of mononuclear cells, was evaluated. LPS stimulated samples showed high levels of the CD14 receptor and injury did not reduce the amount of receptor expression detectable when compared to mononuclear cells of control subjects. Furthermore, co-culture with PGE₂ in mononuclear cells from both control and injured subjects did not markedly alter the levels of CD14 receptor expression.

These results also demonstrate that co-incubation with a selective COX-2 inhibitor, NS-398, after injury reverses the down regulation of the EP2 and EP4 receptor expression.

### EXAMPLE VI

### The Effect of Selective COX-2 Inhibitors on the Immune Response Following Injury in Human Patients

Based on the foregoing data showing the beneficial effects of treatment with a selective cyclooxygenase-2 (COX-2) inhibitor, NS-398, in altering post-injury mediated pathways of infection, human patients suffering from an injury, are treated with a selective COX-2 inhibitor such as Celebrex (celecoxib).

Alternatively, Vioxx is administered to patients pre- and/or immediately post-injury (burn, fracture, hemorrhage, surgery) for 24 hours. By analyzing blood samples from these patients, the amount of pro-inflammatory mediators produced by peripheral blood mononuclear cells is determined. Elevations in these cytokines and transcriptional activating factors have been shown to correlate with the immune suppression and subsequent immune dysregulation, which predisposes subjects to infectious complications.

The following measurements are taken:

1. Serum and peripheral blood monocyte production of cytokines and inflammatory mediators: tumor necrosis factor-alpha (TNF-α, interleukin-6 (IL-6), interleukin-10 (IL-10), and leukotriene-B4 (LTB4);

2. urinary prostaglandin E-2 (PGE-2), standardized by urine creatinine;

3. quantitative amounts of COX-2 mRNA from peripheral blood mononuclear cells, and;

4. activation of transcriptional factors such as, NFκB, NF-IL6 and AP-1 in peripheral blood mononuclear cells.

Patients with burns, major injury, or who are undergoing elective surgery, are administered Vioxx for two days (immediately after injury or post-op, and then once more 24 hours later). Serum and urine is collected from these patients at 3 time points for changes in cytokines and transcriptional factors (immediately after injury, and then at 24 and 72 hours later; elective surgery patients will have 4 time points since there will be a pre-op blood/urine sample obtained in the surgery clinic). Mononuclear cells are also obtained from the blood and assessed for differences in ability to express these cytokines as well as levels of COX-2 messenger RNA (mRNA).

Patients are divided into four groups, burns, major injury, elective surgery, and appropriate controls. They are further divided into two subgroups, placebo and treatment, for a total of eight groups. A total of 120 patients, are included in the study, with 15 patients per each subgroup, as depicted in the diagram below:

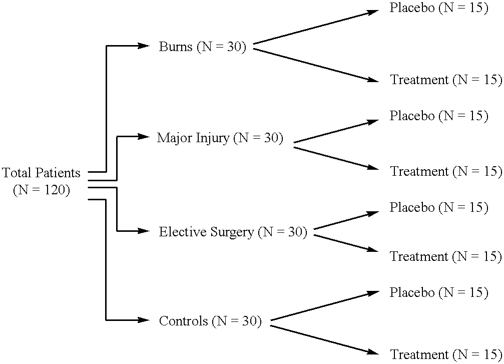

All patients, including controls, range from 18-80 years old (male or female). Controls consist of healthy volunteers (male or female) meeting all of our inclusion/exclusion criteria. Patients have the study fully explained by a qualified IRB listed M.D. and will sign the consent form before beginning the study.

Inclusion Criteria

Burns: Patients who have sustained burns >15% total body surface area.

Major injury: Patients who have sustained a long-bone fracture requiring admission to the hospital for 72 hours, and/or have lost blood (shock class II as per ATLS criteria) from a motor vehicle accident or other trauma.

Elective surgery: Patients undergoing major elective surgery: liver resection, pancreatic surgery, esophageal resection, gastrectomy, colorectal surgery

Controls: healthy volunteers within the age range of 18-80.

Exclusion Criteria

Any patient or control with any evidence of immunosuppression, such as HIV, other immune suppressing conditions/diseases, recent steroid or NSAID use, or chemotherapy/radiation therapy for cancer within the past three months, will be excluded from the study. In addition, patients with conditions not yet fully evaluated for safety with Vioxx, such as pregnancy, severe hepatic insufficiency (total bilirubin >2) and advanced renal insufficiency (creatinine >1.8), are excluded. Also, any patient with a known allergic reaction to NSAIDs, hypersensitivity to Vioxx or any of its components, history of peptic ulcers, or history of gastrointestinal bleeding is excluded.

Experimental Protocol

Patients who have sustained burns or major injury receive Vioxx 50 mg orally, or placebo, as soon as possible after the event, and a second dose 24 hours later (2 doses). The elective surgery patients are given Vioxx 50 mg or placebo 24 hours prior to surgery, another dose two hours after surgery and a third dose 24 hours after surgery (3 doses). Post-operative patients that have nasogastric tubes are given the medicine in oral suspension form. Controls will receive Vioxx 50 mg or placebo upon inclusion in the study, and another dose 24 hours later (2 doses).

Blood and urine samples on admission to the emergency room and at 24 and 72 hours after admission for the burns and major injury patients are obtained. Similarly, blood and urine samples are obtained pre-operatively, immediately post-operation, and at 24 and 72 hours after surgery for the elective surgery patients. Control patients have blood and urine samples obtained upon inclusion in the study, and at 24 and 72 hours after inclusion. Therefore, each patient in the study has blood obtained at three separate intervals for the study. Each blood and urine sample consists of approximately 20 ml of fluid.

Patients enrolled in the study are evaluated for serious side effects of Vioxx, such as gastrointestinal bleeding and allergic reactions, and are removed from the study accordingly.

Laboratory Methods and Procedures

Venipuncture: blood is collected by medical doctors or appropriately certified individuals at the time points stated in the experimental protocol. It is then centrifuged and separated for plasma and peripheral blood monocytes by the Ficoll-Hypaque gradient solution followed by adherence. (21)

Urine collection: 24-hour urine samples are obtained at the time points stated above.

Cytokine analysis: plasma from collected patients is analyzed for the following cytokines: TNF-α:, IL-6, IL-10, and LTB-4, through commercially available enzyme linked immunosorbent assay (ELISA) kits. In addition, peripheral blood monocytes are isolated and studied separately. The isolated monocytes stimulated with lipopolysaccharide are then analyzed for production of cytokines in the same manner (22). Urinary PGE-2 levels are measured by commercially available ELISA kits, and standardized by 24-hour urine creatinine. Inhibition of cytokine production (decreases in cytokine) in the patients receiving Vioxx is thus monitored.

COX-2 mRNA: the mononuclear cells are analyzed for COX-2 mRNA production by the well-established method of reverse-transcriptase polymerase chain reaction (RT-PCR) as described in Example III. Decreases in COX-2 mRNA levels in patients receiving Vioxx are thus monitored.

Transcriptional factor activation: the isolated mononuclear cells are analyzed for NFκB, NF-IL6, and AP-1 transcriptional factor activation by gel-shift or electrophoretic mobility assays (22). These transcriptional factors are known to be activated in inflammatory states. Reduced activity of these factors in patients receiving Vioxx is thus monitored.

### EXAMPLE VII

A patient involved in a motor vehicle accident is brought to an emergency room, having sustained fractures and blood loss and possible organ injury. After appropriate operative management, the patient is brought to the intensive care unit for monitoring. At this time, the patient receives an oral dose of 400 mg Celebrex® twice daily or an oral dose of 50 mg Vioxx® daily. The patient subsequently does not suffer from an overwhelming pro-inflammatory response that may lead to infections complications and leaves the intensive care unit. The patient is then discharged home.

### EXAMPLE VIII

A patient comes into the emergency room with severe mid-epigastric pain that radiates to the back and is diagnosed with acute pancreatitis. The patient is transferred to the intensive care unit for observation and is given an oral dose of 400 mg Celebrex® twice daily and an oral dose of 50 mg Vioxx® daily. The patient subsequently recovers without complication and is discharged home.

### References

1. Lin, E., Calvano, S. E., and Lowry, S. F. Inflammatory cytokines and cell response in surgery. Surgery. 127: 117, 2000.

2. Rangel-Fausto M S et al., (1995) “The natural history of the systemic inflammatory response syndrome (SIRS): a prospective study. JAMA 273:117-23.

3. Rangel-Fausto M S et al., “Increases in National Hospital Discharge Survey Rates for Septicemia—United States, 1979-1987: Morb. Mortal. Wkly Rep. 39:31-4, 1990.”

4. Baker, C. C., Oppenheimer, L., Stephens, B., Lewis, F. R., and Trundey, D. D. Epidemiology of trauma deaths. Am. J. Surg. 140: 144, 1980.

5. Gabay, D., and Kushner, I. Acute-phase proteins and other systemic resonses to inflammation. N. Engl. J. Med. 340: 448, 1999.

6. Zellweger, R., Ayala, A., Zhu, X. L., Morrison, M. H., and Chaudry, I. H. Effect of surgical trauma on splenocyte and peritoneal macrophage immune function. J. Trauma. 39: 645, 1995.

7. O'Sullivan, S. T., Lederer, J. A., Horgan, A. F., Chin, D. H. L., Mannick, J. A., and Rodrick, M. L. Major injury leads to predominance of the T helper-2 lymphocyte phenotype and diminished interleukin-12 production associated with decreased resistance to infection. Ann. Surg. 222: 482, 1995.

8. Grbic, J. T., Mannick, J. A., Gough, D. B., and Rodrick, M. L. The role of prostaglandin E2 in immune suppression following injury. Ann. Surg. 214: 253, 1987.

9. McCarter, M. D., Mack, V. E., Daly, J. M., Naama, H. A., Calvano, S. E. Trauma-induced alterations in macrophage function. Surgery. 123: 96, 1998.

10. Faist, E., Mew, A., Baker, C. C., Strasser, T., Alkan, S. S., and Rieber, P., Heberer, G. Prostaglandin E2 (PGE₂)-dependent suppression of interleukin-2 production in patients with major trauma. J. Trauma. 27: 837, 1987.

11. Ertel, W., Singh, G., Morrison, M. G., Ayala, A., and Chaudry, I. H. Chemically induced hypotension increases PGE₂ release and depresses macrophage antigen presentation. Am. J. Physiol. 264: R655, 1993.

12. Fukushima, R., Alexander, J. W., Wu, J. Z., and Mao, J. X., Szczur, K., Stephens, A. M., Ogle, J. D., Ogle C. K. Time course of production of cytokines and prostaglandin E2 by macrophages isolated after thermal injury and bacterial translocation. Circ. Shock. 42: 154, 1994.

13. Vane, J. R. Inhibition of prostaglandin synthesis as a mechanism oaf action for aspirin-like drugs. Nature (London). 231: 232, 1971.

14. Fletcher, J. R., and Ramwell, P. W. Indomethacin improves survival after endotoxin in baboons. Adv. Prostaglandin. Thromboxane Res. 7: 821, 1980.

15. Hinshaw, L. B., Solomon, L. A., Erdos, E. F., Reins, D. A., and Gunter, B. J. Effects of acetylsalicylic acid on the canine response to endotoxin. J. Pharmacol. Exp. Ther. 157: 665, 1967.

16. Parratt, J. R., and Sturgess, R. M. E. coli endotoxin shock in the cat: treatment with indomethacin. Br. J. Pharmacol. 53: 485, 1975.

17. GI side effects of COX-2 NSAIDS

18. Versteeg, J. J., van Vergen en Henefouwen, P. M. P., van Deventer, S. J. H., and Peppelenbosch, M. P. Cyclooxygenase-dependent signaling: molecular events and consequences. FEBS Lett. 445:1, 1999.

19. Futaki, N., Takanashi, S., Yokohama, M., Arai, I., Higuchi, S., and Otomo, S. NS-398, a new antiinflammatory agent, selectively inhibits prostaglanding G/H synthase/cyclooxygenase (COX-2) activity in vitro. Prostaglandins. 47: 55, 1994.

20. Hinson, R. M., Williams, J. A., and Shacter, E. Elevated interleukin 6 is induced by prostaglandin E2 in a murine model of inflammation: possible role of cyclooxygenase-2. Proc. Natl. Acad. Sci. U.S.A. 93: 485, 1996.

21. Boyum, A., Isolation of mononuclear cells and granulocytes from human blood. Clin. Lab. Inves. Supp. 21:77-79, 1968.

22. Andrews N C and Faller D V, A rapid micropreparation technique for extraction of DNA-binding proteins from limiting numbers of mammalian cells. Nucleic Acids Research 19(9):2499, 1991.

23. Mack, V., McArter, M. D., Naama, H. A., Calvano, S. E., Daly, J. M., Dominance of T-Helper 2-type cytokines after severe injury. Arch. Surg. 31:1303-1309, December, 1996.

What is claimed is:

1. A method of prophylaxis of a patient at risk for systemic inflammatory response syndrome and complications thereof or of treating a patient having systemic inflammatory response syndrome or complications thereof, which comprises administering to said patient a therapeutically effective amount of a selective inhibitor of cyclooxygenase-2.

2. The method of claim 1, wherein the systemic inflammatory response syndrome is at least one of sepsis, pancreatitis, burns, or trauma.

3. A method of prophylaxis of a patient at risk for systemic inflammatory response syndrome and complications thereof or of treating a patient having systemic inflammatory response syndrome or complications thereof, which comprises administering to said patient a therapeutically effective amount of a drug which interferes with binding of PGE₂ to one or more PGE₂ receptors.

4. A method of prophylaxis of a patient at risk for systemic inflammatory response syndrome and complications thereof or of treating a patient having systemic inflammatory response syndrome or complications thereof, which comprises administering to said patient a therapeutically effective amount of a drug which stimulates one or more PGE₂ receptors.

5. The method of claim 3 or 4 wherein the PGE₂ receptors are selected from the group consisting of EP1, EP2, EP3, and EP4.

6. The method of claim 3 wherein the drug is selected from the group consisting of a small molecule, peptide, peptide mimetic, and RNA-DNA-based structure.

7. The method of claim 3, wherein the systemic inflammatory response syndrome is at least one of sepsis, pancreatitis, burns, or trauma.

8. The method of claim 4 wherein the systemic inflammatory response syndrome is at least one of sepsis, pancreatitis, burns, or trauma.

9. The method of claim 1 wherein the patient at risk for systemic inflammatory response syndrome and complications thereof is a patient who has sustained at least one of trauma, burn injury, life threatening blood loss from penetrating injury, or a patient who has undergone surgery.

10. The method of claim 3 wherein the patient at risk for systemic inflammatory response syndrome and complications thereof is a patient who has sustained at least one of trauma, burn injury, life threatening blood loss from penetrating injury, or a patient who has undergone surgery.

11. The method of claim 4 wherein the patient at risk for systemic inflammatory response syndrome and complications thereof is a patient who has sustained at least one of trauma, burn injury, life threatening blood loss from penetrating injury, or a patient who has undergone surgery.

12. The method of claim 1 wherein the complications of systemic inflammatory response syndrome is at least one of septic shock, infections such as pneumonia, septicemia, bacteremia, urinary tract infections, wound infections or drug reactions.

13. The method of claim 3 wherein the complications of systemic inflammatory response syndrome is at least one of septic shock, infections such as pneumonia, septicemia, bacteremia, urinary tract infections, wound infections or drug reactions.

14. The method of claim 4 wherein the complications of systemic inflammatory response syndrome is at least one of septic shock, infections such as pneumonia, septicemia, bacteremia, urinary tract infections, wound infections or drug reactions.

15. The method of claim I wherein the cyclooxygenase-2 inhibitor is at least one of NS-398, celicoxib, MK-0966, or paracoxib.

16. A method of beneficial immune modulation which comprises administering to a patient in need of such modulation a therapeutically effective amount of a drug which stimulates one or more PGE₂ receptors.

17. A method of beneficial immune modulation which comprises administering to a patient in need of such modulation a therapeutically effective amount of a drug which interferes with binding of PGE₂ to one or more PGE₂ receptors.

18. The method of claim 16 wherein the drug is selected from the group consisting of a small molecule, peptide, peptide mimetic, and RNA-DNA-based structure.

19. The method of claim 17 wherein the drug is selected from the group consisting of a small molecule, peptide, peptide mimetic, and RNA-DNA-based structure.

20. The method of claim 16 wherein the drug is at least one of sulprostone, 11-deoxy-PGE₁ or ONO-AP-324.

21. The method of claim 17 wherein the drug is at least one of AH-6809, ONO-8711, ONO-8713, and AH23848.

22. The method of claim 3 wherein the drug is at least one of AH-6809, ONO-8711, ONO-8713, and AH23848.

23. The method of claim 4 wherein the drug is at least one of sulprostone, 11-deoxy-PGE₁ or ONO-AP-324.
